(19) United States
(12) Patent Application Publication
(10) Pub. No.: US 2003/0115624 A1
(43) Pub. Date: Jun. 19, 2003

(54) Quantitative trait loci for insect resistance in soybeans

(76) Inventors: L. Irene Terry, Park City, UT (US); Kevin Chase, Salt Lake City, UT (US)

Correspondence Address: GREENLEE WINNER AND SULLIVAN P C, 5370 MANHATTAN CIRCLE, SUITE 201, BOULDER, CO, 80303, US

(21) Appl. No.: 09/803,676

(22) Filed: Mar. 9, 2001

Related U.S. Application Data
(60) Provisional application No. 60/188,439, filed on Mar. 10, 2000.

(51) Int. Cl. A01H 5/00; C12Q 1/68; C07H 21/04; C12N 15/82

(52) U.S. Cl. 800/260; 435/6; 800/279; 800/312; 536/23.6

(57) ABSTRACT

This invention relates to a method of breeding plants to improve a trait of agronomic or horticultural value and plants produced by the method. The invention particularly relates to a method of plant breeding to affect an interaction between a plant and an interacting organism. Interactions that can be modified or affected are the positive beneficial, the negative or deleterious, or a harmless interaction. The breeding method of the invention provides progeny plant line phenotypes toward interacting organisms that exceed the range of phenotypes displayed by the parental strains. The methods of the invention can be implemented with the aid of molecular markers for quantitative trait loci.

### CROSS-REFERENCE TO RELATED APPLICATION

This application claims priority from U.S. Provisional application No. 60/188,439, filed Mar. 10, 2000, which is incorporated herein by reference to the extent not inconsistent herewith.

### STATEMENT REGARDING FEDERALLY SPONSORED RESEARCH AND DEVELOPMENT

The research leading to the invention was supported in part by U.S. Department of Agiculture National Research Institute grants 93-37302-9180 and 96-35302-3638. The U.S. government has certain rights in the invention.

### TECHNICAL FIELD OF THE INVENTION

The invention relates to a method of breeding plants. The breeding methods allow for the identification of quantitative traits of agronomic value and the subsequent introduction of such traits into other plant lines. The breeding methods advantageously correlate traits of agronomic value with specific genetic loci using a set of molecular markers. The molecular markers are used to follow the introgression or breeding of the linked traits into other plant lines.

### BACKGROUND OF THE INVENTION

Plant breeders of field crops are primarily concerned with agronomic traits such as plant height, germination time, time to maturity, crop yield, resistance to disease, resistance to environmental stress and the like. Such traits are seldom controlled by a single gene with an all or nothing effect. Instead, the traits are typically controlled by genes whose effects are marginally quantitative, i.e., their effects are observable primarily by comparing quantitative measurements of specific traits and observing an increment or decrement of the trait. The locus of such a trait on a genetic map is therefore termed a quantitative trait locus (QTL). The term QTL is sometimes used synonymously to mean the gene affecting the trait.

Identifying specific QTLs, mapping them and understanding the genetic and molecular basis for their action was largely impossible prior to the development of molecular markers. Molecular markers are a type of phenotype which can be detected by molecular techniques such as hybridization to a labeled DNA probe. Types of molecular markers include RFLP (restriction fragment length polymorphism), SSR (simple sequence repeat) markers, isozyme markers and the like. For a review, see Dudley, J. W. (1993) Crop. Sci. 33:660-668.

The power of molecular markers lies in the large number that can be generated and in the ease and rapidity with which they can be measured. Prior to the advent of RFLP markers, only 17 linkage groups covering approximately 420 centimorgans (cM) had been identified in soybean using 57 classical markers such as flower color, seed coat color, seed coat peroxidase, root fluorescence, specific pest resistances and the like [Palmer, R. G. et al. (1987) Qualitative Genetics and Cytogenetics, In Wilcox, J. R. (ed) Soybean Improvement, Production and Uses 2nd Ed. Agronomy 16:145-199]. In contrast, Keim et al. (1990) Genetics 126:735-742, constructed a genetic map that included 130 RFLP markers in 26 linkage groups, covering approximately 1200 cM. Later, Diers et al. (1992) Theor. Appl. Genet. 83:608-612 expanded the map to 252 markers in 31 linkage groups, covering 2147 cM. Libraries of molecular marker probes and PCR primers have been made for most agronomically important species in addition to soybean including tomato, maize, wheat and barley. The genetic maps of all such species have been significantly improved by the use of molecular markers.

Molecular markers have proven to be of great value for increasing the speed and efficiency of plant breeding. Most traits of agronomic value, e.g., pest resistance, yield, and the like, are difficult to measure, often requiring a full growth season and statistical analysis of field trial results. Interpretation of the data can be obscured or confused by environmental variables. Occasionally, it has been possible for breeders to make use of conventional markers such as flower color which could be readily followed through the breeding process. If the desired QTL is linked closely enough to a conventional marker, the likelihood of recombination occurring between them is sufficiently low that the QTL and the marker co-segregate throughout a series of crosses. The marker becomes, in effect, a surrogate for the QTL itself. Prior to the advent of molecular markers, the opportunities for carrying out marker-linked breeding were severely limited by the lack of suitable markers mapping sufficiently close to the desired trait. Map distance is simply a function of recombination frequency between two markers, QTLs or markers and QTLs. Consequently, if a marker and a QTL map too far apart, the recombination frequency will be higher such that the marker becomes no longer associated with the QTL during a series of crosses or self-pollinations. Having a wide selection of molecular markers available throughout the genetic map provides breeders the means to follow almost any desired trait through a series of crosses, by measuring the presence or absence of a marker linked to the QTL which affects that trait. The primary obstacle is the initial step of identifying a linkage between a marker and a QTL affecting the desired trait.

Molecular markers provide two additional operational advantages. First, since they exist as features of the plant DNA itself, they can be detected soon after germination, for example by analysis of leaf DNA of seedlings. Selection for plants carrying the marker can be performed at the seedling stage, thereby saving the space and energy formerly needed to grow large numbers of plants to maturity. Second, molecular markers do not depend on gene expression for detection. Their use is unlikely to lead to misleading results, such as can occur when environmental or other variables modify expression of conventional marker genes.

Identifying specific markers with specific traits of agronomic value remains a problem. In particular, identifying QTLs with linked markers has only become possible with the availability of a large number of molecular markers to which QTLs can be linked [Dudley (1993)]. An important tool for evaluating quantitative traits and linkage to molecular markers is the recombinant inbred (RI) population. RI populations include individual lines representing stable inbred (therefore homozygous) segregant progeny from a single genetic cross [Burr, B. et al. (1988) Genetics 188:519-526; Carillo, J. M. et al. (1990) Theor. AppL. Genet. 79:321-330]. An RI population is begun by a cross between two parent inbred (homozygous) cultivars. The two parental cultivars are preferably chosen so that between them a large amount of allelic variation exists. Only those molecular markers that are polymorphic between the two cultivars, i.e., have a distinguishable difference between the two cultivars, can be used. Therefore, the greater the overall allelic variation between cultivars, the greater will be the number of usable molecular markers. The individual progeny of the first cross are then selfed for several generations. The selfing process occurs naturally in soybean, which is a self-pollinator. In order to ensure equal representation from all individuals of the cross, segregants are maintained separately during subsequent generations, a process known as single seed descent. During several generations of selfing, the progeny tend toward homozygosity at each locus, although each locus could have originated from either one of the original parent cultivars. As a result, the population eventually contains genes that are mostly homozygous at each locus, but which are randomly mixed as to parental source in a multiplicity of combinations depending on recombination events. After eight generations of selfing, the theoretical frequency of heterozygosity is 1 in 2⁸. Therefore, the RI population is essentially homozygous. As a result, each segregant in the RI population can be analyzed en masse in repeated experiments measuring various traits of agronomic interest. Simultaneously, allelic variation of individual molecular markers can be determined. It should be possible, in principle, to analyze such data for correlations between specific traits and specific marker alleles and thus identify QTLs linked to marker alleles.

QTLs can interact with one another additively and epistatically. Additive interactions are those in which the effect on plant phenotype of the presence of a given pair of QTLs in the same plant is the sum of their individual effects measured in plants in which each QTL is present alone.

“Epistasis” is the genetic term used to denote situations where non-allelic genes interact non-additively to affect the expression of a phenotype. Classical epistatic effects are observed, for example, between pigment genes and genes affecting pigment distribution. Where a gene for pigment synthesis is altered or inactivated, expression of a pigment distribution gene may not be observable. Quantitative traits in plants are the result of interactions between multiple QTLs and the environment [Tanksley, S. O. (1993) Ann. Rev. Genet. 27:205-233]. The existence of epistasis among QTLs extends the complexity and difficulty of identifying molecular markers linked to QTLs.

The desirability of generating plant lines resistant to various interacting organism is a continual goal of breeding programs. Prior art methods for finding resistant germplasm have included screening or searching for resistance in: locally adapted cultivars, foreign accessions/cultivars, natural hybrids, and wild relatives that can be interbred into the crop species. Newer methods involve the engineering of foreign genes or DNA into plant lines that when expresed in the trangenic plants, confers resistance. Examples include genes or DNA from bacterial toxins (Bacillus thuriengiensis toxins), viruses, etc., and/or regulating genes to control plant gene expression. The method of breeding of the invention uses cultivars from genetically different backgrounds (e.g., those from diverse ecological geographical settings), that are non-resistant, as parents to create an RI population that can be tested for transgressive segregation with respect to resistance/susceptibility in their homozygous offspring. Traits can be correlated with specific molecular markers in these populations and particularly beneficial alleles can be identified bred into other plant lines.

Other methods designed to identify QTLs associated with resistance have focused on using plants which have a resistance trait already known. The use of >F2 lines aids the search for markers associated with the resistance. Markers associated with insect resistance in tomato, wheat, corn, rice and potato have been found. Specific plant crosses have been examined to determine QTLs associated with plant chemistry, morphology, etc., traits in lines already know to carry resistance. However, quantitative traits that may confer resistance, that can be found in RI segregants through additive and epistatic effects resulting from recombination during crossing are not accessible through such prior art methods.

Lark, K. G. et al. (Proc. Nat. Acad. Sci. USA 92:4656-4660) reported the identification of certain epistatic QTLs in soybean where trait variation at one locus was conditional upon a specific allele at another, in an RI population obtained from a cross between cultivar ‘Minsoy’ and cultivar ‘Noir 1.’ To identify such pairs of loci, the authors chose as the first locus a QTL that had been found to be associated with a measured trait, such as plant height. They then scanned through height data relating to unlinked loci, dividing the population of RI lines into pairwise combinations of the first locus and a second locus. Since, at each locus, there were two identifiable alleles, each pairwise combination actually resulted in four possible combinations:

1) Locus 1 from ‘Minsoy’ and Locus 2 from ‘Minsoy’

2) Locus 1 from ‘Minsoy’ and Locus 2 from ‘Noir 1’

3) Locus 1 from ‘Noir 1’ and Locus 2 from ‘Minsoy’

4) Locus 1 from ‘Noir 1’ and Locus 2 from ‘Noir’

Since the molecular markers used were known to be polymorphic between ‘Minsoy’ and ‘Noir,’ each of the four possible combinations could be identified and scored for distribution of plant heights. Instead of graphing the data as conventional distribution curves (number of plants with a given height on the ordinate vs height on the abscissa), the data were graphed as cumulative distributions. Height was graphed on the abscissa against the rank of each plant from shortest to tallest on the ordinate. Both the positions of the resulting distribution curves and their shape were indicators of interaction. In this way, several loci were identified which, alone, had no apparent effect on plant height but one allele of which did affect the height controlled by another locus. The maximum plant height controlled by a given first gene was therefore conditional upon the simultaneous presence in the plant of the proper allele of a second gene. An interaction of the same type was reported for yield, which was distinguishable from effects on plant height and on maturity. The analytical methods described by Lark et al (1995) and Chase, K. et al. (1997), Theor. Appl. Genet. 94:724-730, incorporated herein by reference, were employed in the present invention. A detailed description appears infra in the Examples.

Other epistatic QTLs affecting soybean yield were disclosed in WO98/49887 published Nov. 12, 1998 and incorporated herein by reference. The authors thereof were able to demonstrate the epistatic interactions by measuring various morphologic traits (e.g., plant height, seed number, seed weight) and growth habit (e.g. time to maturity) as well as overall yield, in field trials of RI lines. However, there are many agronomic traits which are not accessible to evaluation by direct measurement of individual plants. Traits such as pest resistance or pathogen resistance cannot be assessed by direct measurements on individual plants or RI lines in a field trial because of the lack of knowledge of the biochemical basis for the resistance and the impracticability of introducing the pest or pathogen to each plant in a controlled manner. The present invention extends the previous work beyond its limitations by demonstrating that QTLs can be identified and associated with closely linked markers, using measurements made under controlled laboratory conditions. In particular, the existence of QTLs and associated markers affecting a plant's interaction with another organism, such as a pathogen or insect pest, can be determined and evaluated by measurements of the interacting organism, rather than the plant itself.

### SUMMARY OF THE INVENTION

The present invention provides a method for plant breeding to improve a quantitative trait of agronomic value. The method entails identifying a molecular marker linked to a quantitative trait locus (QTL) at least one allele of which has an effect on a quantitative trait. The method includes means for identifying a plant QTL affecting an interaction between the plant and another organism by assessing the plant's effects on the interacting organism. The interacting organism can be any organism, most commonly an insect or microbial organism. The interaction can be either harmful or beneficial to the plant. The invention is exemplified by identification of QTLs in soybean that affect the plant's resistance to an insect pest. However, it will be understood by those skilled in the art that the invention can be applied, in principle, to other plant species and to other interacting organisms by adapting the teachings herein to the desired plant species and interacting organism, as needed.

Measurements of the interacting organism are made for parameters that can be correlated with the plant interaction. As demonstrated herein, for example, plant resistance to insect damage can be assessed by measurements of larval growth, pupal weight, development rate and nutritional efficiency. By assessing a variety of parameters, the range of plant responses and the quantitative interactions between them can be more fully explored.

Markers linked to QTLs having either additive or epistatic interactions can be identified by correlating the presence of a given marker allele with the observed response. Conventional breeding methods can be employed to introduce one or more marked loci into other varieties or cultivars, as desired, and to select for the presence of the desired QTLs through successive rounds of crossing and selection. The breeding process can therefore result in a novel and unique plant variety distinguished from a first parent variety by having a gene that affects a quantitative trait that provides improvement in a trait of agronomic value. Further, other genes affecting the same quantitative trait can be introduced, and the newly introduced genes can be those which interact additively or which interact epistatically. At least one such allele of such genes is contributed by a second plant variety. As is well-known in the art, the use of a molecular marker linked to the quantitative trait locus as a surrogate for the trait itself makes it possible to identify the progeny plants bearing the desired allele of the desired QTL. The closer the association between the marker and the QTL, the greater the likelihood that the marker will not be segregated from the QTL by a recombination event during multiple crosses.

The process of the invention is characterized by the following general stages. RI soybean lines are prepared, as known in the art and described herein. Assay methods are developed for measuring putative effects which the plant may have on the interacting organism. Using the developed assay methods, the plant-organism interactions are measured for a wide range of RI lines. Where the measured parameters reveal a pattern of consistency within individual plants of the same RI line and variability over the range of RI lines, the variability indicates that different plant alleles have different effects on the interacting organism. By correlating the existence of such measured differences with the specific molecular markers, QTLs affecting the plant-organism interaction are identified. QTLs associated with the greatest range of variation among RI lines are likely to have the greatest quantitative effect. The linkage distance between the marker and the QTL can be estimated by statistical methods described herein and known in the art. The parental source of the desired QTL allele can be identified by the techniques described herein or known in the art. The desired allele can then be introduced into any desired commercial strain, using conventional plant breeding methods.

The invention can be applied for identification of any plant QTL affecting an interaction between the plant and another organism. Thus, any plant variety that can be propagated by conventional hybridization can be bred to create RI populations from which RI lines can be grown and maintained. Molecular marker libraries can be developed, and for many agronomic crop plants, including corn, soybean and tomato, such libraries already exist. Interacting organisms can be bacterial, viruses, fungi, insects, even other plants. While interactions that lead to increased resistance to a pathogen are obvious applications, there are known beneficial interactions (nodulation, pollination, soil nutrient release and the like) which can be regulated by plant QTLs also. The type of measurement of the interacting organism is tailored to any measurable parameter that might be affected by the plant, depending on known biological and biochemical features of the interaction. As exemplified here for an insect pest of soybean, measurement of average larval weight at 12 days, pupal weight, development rates, survival and nutritional indices of insects reared on excised leaf tissue from a set of RI lines revealed substantial variations between the most resistant and the most susceptible RI lines, over a 5-fold range in some cases. By measuring a variety of parameters, plant QTLs affecting a variety of plant strategies for modifying interacting organisms are revealed. Typical parameters of the interacting organism that can be measured include, but are not limited to, measurements of growth, viability, development rate, reproductive rate, nutritional efficiency, behavior and the like, as will be apparent to those skilled in the art.

As a result of the invention molecular markers were identified for soybean QTLs affecting insect resistance. Significant association with one or more QTLs affecting the test insect are given in Table 7. In addition, all of the foregoing were significantly associated with measurements of development: weight and weight gain over several days, and development rates to prepupa and to pupa stage. Measurements of pupal weight were also correlated with some of the same markers. Cumulative mortality was significantly associated with markers R013, Satt507 and Satt575. The results demonstrate that various QTLs affect soybean resistance to insects by a variety of mechanisms, which are reflected in the differences observed among the variety of parameters measured. Many of the QTLs identified also affected the plant's interaction with other insect species as well.

From these results, and like results obtainable by extension of these data to other RI lines, other insect species, other interacting organisms, other molecular markers, and other plant varieties, etc., molecular markers for introducing greater insect resistance into soybean, or for introducing any desired trait affecting any interacting organism with a plant, can be obtained. The foregoing marker set and others identified in like manner are useful as tools for breeding desired traits into plants, according to principles and methods well known in the art.

In an embodiment of the invention, alleles of QTLs identified by the breeding methods described herein can be bred into other plant varietals which lack such QTLs. QTLs identified by the invention can be bred into compatible cultivars or plant lines. QTLs identified by the invention can be bred into transgenic plants. QTLs identified by the invention can be bred into non-genetically modified organisms. QTLs identified by the invention can be expressed or introduced into other plant varietals or lines by other means available to those of ordinary skill in the art, e.g., through genetic engineering technology.

The invention is exemplified by the demonstration of QTLs and molecular markers linked thereto which affect resistance of soybean to corn earworn. In particular, the following molecular markers were identified as linked to QTLs affecting interactions between soybean and interacting organisms: Satt192, Satt285, Satt301, Satt302, Satt353, Satt507, Satt531, Satt575, Satt676, Sct_{—}046, and Sat_{—}112. Primers for these markers (F for foward, R for reverse) are given below.

| | |
|---|---|
| Satt192: | |
| F = CACCGCTGATTAAGATTTTT | SEQ ID NO:1 |
| R = CGCTGAGTTGTTTTCATC | SEQ ID NO:2 |
| Satt_285: | |
| F = GCGACATATTGCATTAAAAACATACTT | SEQ ID NO:3 |
| R = GCGGACTAATTCTATTTTACACCAACAAC | SEQ ID NO:4 |
| Satt301 | |
| F = GCGAAACACTCCTAGTTGATTACAAA | SEQ ID NO:5 |
| R = GCGATATAATGCACAAAGAAATTAAAGA | SEQ ID NO:6 |
| Satt302 | |
| F = GCGAACTGTAGTTTACTAAAAATAAGTG | SEQ ID NO:7 |
| R = GCGGACTGAATTAATATTGGTGTTGAATT | SEQ ID NO:8 |
| Satt353 | |
| F = CATACACGCATTGCCTTTCCTGAA | SEQ ID NO:9 |
| R = GCGAATGGGAATGCCTTCTTATTCTA | SEQ ID NO:10 |
| Satt507 | |
| F = GCGCTCAGCCTTGTTAAATCACTT | SEQ ID NO:11 |
| R = GCGCTACTCTCGTGTCGTTAGTTA | SEQ ID NO:12 |
| tt531 | |
| F = GCATGCAACTGAGGGAGCAGAT | SEQ ID NO:13 |
| R = GCCACAAATTATGCAGAATATA | SEQ ID NO:14 |
| Satt575 | |
| F = GCGGCTAATTTTGTTTATAGGAAT | SEQ ID NO:15 |
| R = CCGCTACCATCTCGGAGGACT | SEQ ID NO:16 |
| Sat_112 | |
| F = TGTGACAGTATACCGACATAATA | SEQ ID NO:17 |
| R = CTACAAATAACATGAAATATAAGAAATA | SEQ ID NO:18 |
| Sct_046 | |
| F = AAAAAGGAAACTTCGTCA | SEQ ID NO:19 |
| R = AAACTAAACAGTGTCCATAAGA | SEQ ID NO:20 |

It is contemplated that the breeding methods of the invention can be used in a variety of plants such as grasses, legumes, starchy staples, Brassica family members, herbs and spices, oil crops, ornamentals, woods and fibers, fruits, medicinal plants, and alternative and other crops. Preferably the invention can be used in plants such as sugar cane, wheat, rice, maize, potato, sugar beet, cassava, barley, soybean, sweet potato, oil palm fruit, tomato, sorghum, orange, grape, banana, apple, cabbage, watermelon, coconut, onion, cottonseed, rapeseed, and yam.

### BRIEF DESCRIPTION OF THE FIGURES

FIG. 1. An example of the 12-day larval weight results from one Helicoverpa zea bioassay, in which the parents of the RI population, Minsoy and Noir 1, are included. The results demonstrate the intermediate effect of the parental plant defense phenotypes and some of the extreme RI phenotypes, including the resistant, RI-4, and susceptible, RI-55, standards.

FIG. 2. Cumulative distributions of Helicoverpa zea a) 12 day larval weight; b) Pupal weight; and c) Percent survival to the pupal stage when reared on different RI lines, averaged over all bioassays. The parent lines, Minsoy and Noir 1, resistant lines, RI-272 and RI-4, and susceptible lines, RI-16 and RI-55, are indicated.

FIG. 3. Segregation of QTLs associated with larval weight at the 12th day of development as shown by the cumulative frequency distribution of RI values; sub-populations correspond to alleles at Sat_{—}112 (A) or Satt575 (C) on linkage group U2 or at Satt302 on linkage group U10 (B and D). Data for the Minsoy X Noir 1 population are presented in panels (A) and (B) and for the Minsoy X Archer population in panels (C) and (D). Parental values are indicated.

FIG. 4. Composite MN and MA genetic maps of the relevant portions of the linkage groups with significant QTLs for corn earworm larval development traits. The symbols to the left of the marker names indicate the mapping populations in which the marker segregated (i.e., was polymorphic). The number to the right of the marker names indicates the map distance to the next marker (in centimorgans). Markers listed in Table 2 are indicated in bold. Linkage groups are labeled with both the Utah names and in parentheses, e.g. (J), the Iowa names.

FIG. 5. Simple interval mapping of LOD scores associated with corn earworm larval development traits for linkage groups U2, U8, U9, U10, and U11 in both RI populations. Linkage groups are drawn to scale. The linkage group position (x-axis) is graphed against the LOD score (y-axis) for each population. A threshold line for significant QTL at a LOD score of 2.5 is presented. The first column of graphs represents the linkage groups with significant QTLs in the MN population only, the second column, those with significance in both RI populations, and the third, those with significance in the MA population only. Markers are labeled along the x-axis of each linkage group and correspond to the markers and distances listed in FIG. 4.

### DETAILED DESCRIPTION OF THE INVENTION

The following terms are used as defined herein:

Quantitative Trait—a trait which displays a continuous range of variation over a number of different plant varieties. The variation is considered to be affected by a plurality of genes. The genes controlling quantitative traits are considered to control incremental changes of the variation, and may interact with one another. By their nature, quantitative traits can have an effect that is only indirectly related to their primary function. For example, a gene controlling the length of maturation time can also be identified as affecting plant height, since the plant will continue to grow throughout the maturation period. Environmental interactions also play an important part in measurement of a quantitative trait. For example, a trait such as yield will be affected by a trait of nematode resistance, in nematode-containing soils.

Interacting organism is a term to describe another organism with which the plant interacts. Such interactions may be beneficial, negative or deleterious, or harmless. Particular interacting organisms include pest insects. Pest insects are: beetles including, but not limited to, bean leaf, blister, spotted cucumber, grape colaspis, Japanese and Mexican bean; caterpillars including, but not limited to, armyworms (beet and yellow striped), corn earworms, green cloverworm, soybean looper, velvetbean; Mexican bean beetle larva; stink bugs including, but not limited to, brown and green/Southern green; orange colaspis larvae; soybean stem borer; three-cornered alfalfa hopper. Other interacting organisms include, but are not limited to, viruses, bacteria, fungi and any other such organism.

Quantitative Trait Locus (QTL) is an operational term used to denote a region of the plant genome that can be associated with a quantitative trait. The term QTL is sometimes used synonymously with the gene affecting the trait. However, since QTLs are identified by linkage to molecular markers, the “locus” is more accurately described as the segment of genome remaining linked to the marker through a series of generations while continuing to affect the trait (under appropriate conditions). Physically that segment will include the gene but can also include flanking DNA.

Linkage is defined by classical genetics to describe the relationship of traits which co-segregate through a number of generations of crosses. Genetic recombination occurs with an assumed random frequency over the entire genome. Genetic maps are constructed by measuring the frequency of recombination between pairs of traits or markers. The closer the traits or markers lie to each other on the chromosome, the lower the frequency of recombination, the greater the degree of linkage. Traits or markers are considered herein to be linked if there is less than 1/10 probability of recombination per generation. A 1/100 probability of recombination is defined as a map distance of 1.0 centiMorgan (1.0 cM).

Molecular marker is a term used to denote a DNA sequence feature which is sufficiently unique to characterize a specific locus on the genome. Examples include restriction fragment length polymorphisms (RFLP) and single sequence repeats (SSR). RFLP markers occur because any sequence change in DNA, including a single base change, insertion, deletion or inversion, can result in loss (or gain) of a restriction endonuclease recognition site. The size and number of fragments generated by one such enzyme is therefore altered. A probe which hybridizes specifically to DNA in the region of such an alteration can be used to rapidly and specifically identify a region of DNA which displays allelic variation between two plant varieties. SSR markers occur where a short sequence displays allelic variation in the number of repeats of that sequence. Sequences flanking the repeated sequence can serve as polymerase chain reaction (PCR) primers. Depending on the number of repeats at a given allele of the locus, the length of the DNA segment generated by PCR will be different in different alleles. The differences in PCR-generated fragment size can be detected by gel electrophoresis. Other types of molecular markers are known. All are used to define a specific locus on the soybean genome. Large numbers of these have been mapped. Each marker is therefore an indicator of a specific segment of DNA, having a unique nucleotide sequence. The map positions provide a measure of the relative positions of particular markers with respect to one another. When a trait is stated to be linked to a given marker it will be understood that the actual DNA segment whose sequence affects the trait lies within about 10 cM of the marker. More precise and definite localization can be obtained if markers are identified on both sides of the QTL. By measuring the appearance of the marker(s) in progeny of crosses, the existence of the QTL can be detected by relatively simple molecular tests, without actually evaluating the appearance of the trait itself, which can be difficult and time consuming.

Epistasis is a term referring to an interaction of two genes where the result is other than the sum of the effects attributable to each gene acting in the absence of the other. An additive effect is not epistatic. In epistasis, the mechanism of the interaction is not taken into account. Consequently, even if no effect can be observed for one or both genes alone, an effect dependant on the presence of both is termed epistatic.

Varietal parent is a term used herein to denote one of two parents of a crossing program. The parental plant varietals may be a commercial varietal or any other plant line. The crossing program is intended to introduce a specific locus or QTL or combination thereof into a commercial variety or other plant line. Various commercial varieties have been developed for optimal performance under specific climate and soil conditions. Often it will be the case that new genes are to be introduced from an extraneous non-adapted or non-commerical line into an existing commercial variety. Through repeated backcrossing and selection the desired loci can be introgressed into the commercial variety while retaining most of the genetic background and performance characteristics of the commercial variety. The variety into which the new genes or loci are to be introduced is termed the varietal parent herein. The variety, line or strain from which the new genes or loci are derived is termed the donor variety. For example, a donor strain can be a non-commercial inbred such as Noir 1.

Agronomic trait is used herein as generally understood in the art to refer to traits or trait combinations which have the effect of making a plant variety valuable as a crop. Common examples of agronomic traits include crop yield, pathogen resistance, insect resistance, drought tolerance, nematode resistance, resistance to lodging and various adaptations to different climate and soil environments such as early maturity for northern climates, heat tolerance for southern climates, and various market-driven qualities such as seed protein content, oil content, color, flavor and the like. The foregoing list is exemplary and not exhaustive, as will be understood in the art. Desirable agronomic traits can be expressed as ratios of quantitative traits as for example maturity/height, yield/height, yield/maturity, height/maturity and the like.

The invention relates to a method of plant breeding that improves the ability of progeny plants to interact with another organism. Plants interact with other organisms in a variety of ways. In soybeans and similar crops of economic importance, interactions deleterious to the soybean crop occur with a host of interacting organisms. In soybeans, such interacting organisms that produce deleterious or negative effects include beetles, caterpillars, stink bugs, nematodes (like the soybean cyst nematodes, root-knot nematodes and any other nematodes) and other soybean diseases. Soybean diseases may be viruses, bacteria, and fungi. The invention preferably produces progeny plant lines which diminish a deleterious or negative interaction with a plant-pest interacting organism. The identification of QTLs through the breeding method of the invention allows for the production of plant lines or cultivars that comprise such QTLs. The introgression of such QTLs into other plant lines or cultivars may be performed by art recognized breeding methods available to one of ordinary skill in the art. The invention furthermore preferably produces progeny plant lines which increase or augment a beneficial interaction between plants of the line and an interacting organism.

The breeding method of the invention is characterized by the following stages. Recombinant inbred plant lines are prepared, as known in the art and described herein. Preferably the RI lines are prepared from two parental lines or cultivars that have a substantial amount of polymorphism between them, i.e., the parental lines are genetically distinct. Assay methods are developed for measuring putative effects which the plant may have on the interacting organism. Using the developed assay methods, the plant-organism interactions are measured for a wide range of RI lines. Variability in measured parameters of the plant-interacting organism assays are assessed for consistency within individual plants of the same RI line and variability over the range of RI lines. Consistency among individuals of a particular RI line indicates that the measured parameters may be correlated to the genetic make-up of the RI line as opposed to external variables such as environment. Variability among different RI lines for the parameters measured in the plant-interacting organism assays indicates that different plant alleles have different effects on the interacting organism. The existence of such measured differences can be correlated with specific molecular markers, and QTLs affecting the plant-organism interaction can be identified. QTLs associated with the greatest range of variation among RI lines are likely to have the greatest quantitative effect. The linkage distance between the marker and the QTL can be estimated by statistical methods described herein and known in the art. The parental source of the desired QTL allele can be identified by the techniques described herein or known in the art. The desired allele can then be introduced into any desired commercial strain, using conventional plant breeding methods.

The invention can be applied for identification of any plant QTL affecting an interaction between the plant and another organism. Thus, any plant variety that can be propagated by conventional hybridization can be bred to create RI populations from which RI lines can be grown and maintained. Molecular marker libraries can be developed, and for many agronomic crop plants, including corn, soybean and tomato, such libraries already exist. Interacting organisms can be bacterial, viruses, fungi, insects, even other plants. While interactions that lead to increased resistance to a pathogen are obvious applications, there are known beneficial interactions (nodulation, pollination, soil nutrient release and the like) which can be regulated by plant QTLs. The type of measurement of the interacting organism is tailored to any measurable parameter that might be affected by the plant, depending on known biological and biochemical features of the interaction. As exemplified here for an insect pest of soybean, measurement of average larval weight at 12 days, pupal weight, development rates, survival and nutritional indices of insects reared on excised leaf tissue from a set of RI lines revealed substantial variations between the most resistant and the most susceptible RI lines, over a 5-fold range in some cases. By measuring a variety of parameters, plant QTLs affecting a variety of plant strategies for modifying interacting organisms are revealed. Typical parameters of the interacting organism that can be measured include, but are not limited to, measurements of growth, viability, development rate, reproductive rate, nutritional efficiency, behavior and the like, as will be apparent to those skilled in the art.

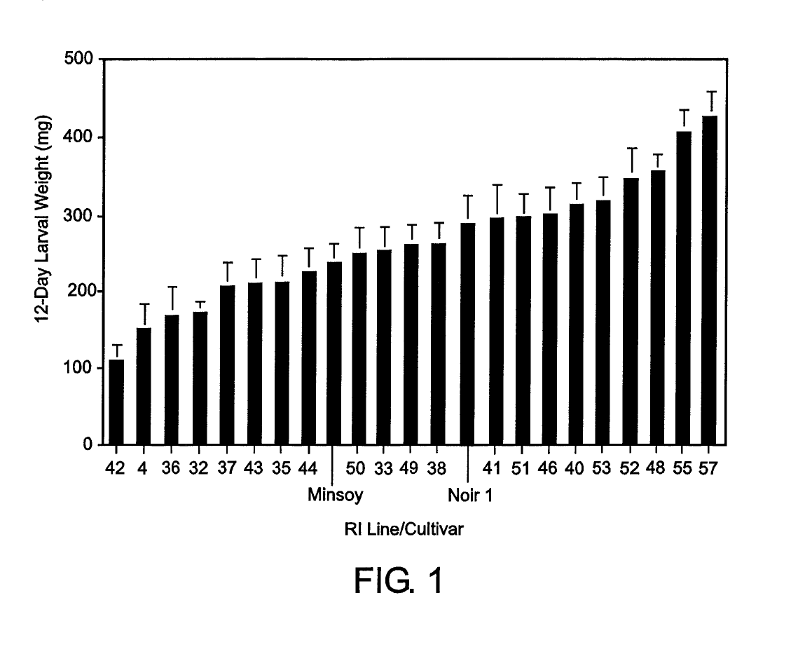

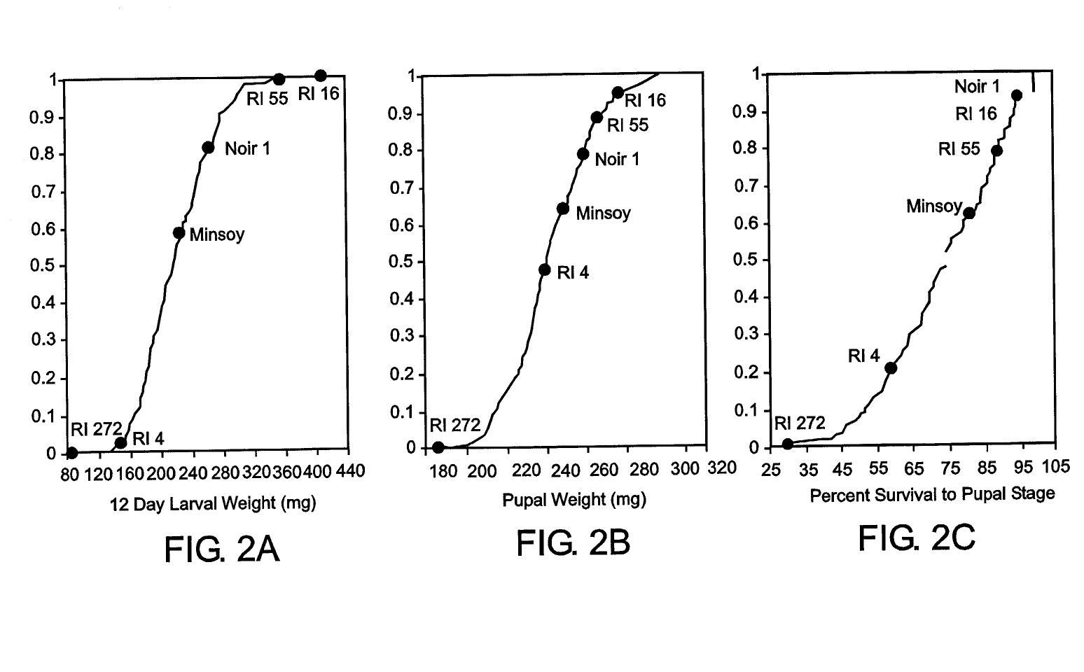

A significant pest of economic importance to soybean crops is the corn earworm, Helicoverpa zea. Soybean plants produced according to the method of the invention were exposed to the corn earworm and assayed for interaction in Helicoverpa zea bioassays. Helicoverpa zea represents a good organism to model pest-soybean interactions. In these studies a population of soybean RI lines were produced from a cross of genetically distinct parental lines: Noir 1 from hungardy and Minsoy from china. The RI and parental lines were mapped with over 500 molecular markers. The affect of various RI lines and the parental lines on Helicoverpa zea were assayed. FIG. 1 presents the results of a typical larval weight bioassay. Each such bioassay contained between 18 and 24 cultivars; i.e., the RI lines to be tested, two standards and sometimes the parents. As can be seen, the range of larval weight variation was large (4-fold). Most assays of larval weight produced this range, but occasionally the variation was large as 8-fold. When data were averaged over all tests, the range of larval weight at 12 days was 85 mg for RI-272 to 418 mg for RI-16 (FIG. 2a, Table 1). Only data from extreme lines of the 240 RI and the parent lines are shown in Table 1, but values for all RI are presented in FIG. 2. In all larval weight bioassays, the parental lines were intermediate, significantly less than the extreme RI lines.

The range of pupal weights was lower, between 185 mg for RI-272 and 308 mg for RI-327 (FIG. 2b, Table 1). Among the lowest pupal weight groups, many pupae were deformed. Again, the parental lines were intermediate in their effect on pupal weight.

The parental lines were also intermediate for two indices of nutrition and as well as for larval developmental rates (Table 1). However, the parent, Noir 1, had little effect on mortality, the % survival to the pupal stage of larvae reared on Noir 1 was among the highest of all lines tested (FIG. 2c, Table 1).

Unexpectedly, the data in Table 1 and FIG. 2 demonstrate that resistance to H. zea arises in many of the RI lines and for most parameters is much more extreme than either of the parents.

Correlations among traits. Table 2 presents some significant correlations between traits. The most striking feature is that survival to the pupal stage is correlated with the parameters of larval growth (e.g., 12 day weight, larval developmental rates). Percent survival to the 12^{th} day of larval growth is not well correlated with larval growth traits. Thus, effects of diet (different RI cultivars) on larval growth translate into mortality primarily after 12 days, during the last larval instars and the period immediately prior to pupation. These correlations indicate that the plant breeding methods of the invention can be used to produce progeny plant lines that increase insect mortality during the last larval instars and the period prior to pupation.

Comparison of resistant and susceptible RI phenotypes with resistant USDA PI lines. Several PI lines (PI-171451, PI-227687 and PI-229358) have been noted for their resistance to soybean insects (see Introduction). The resistance of several RI lines was compared to these PI cultivars (Table 3) using H. zea larval weight, developmental rates, pupal weights, and other nutritional measures as indicators. In every test, larvae reared on PI-227687 weighed significantly more than those reared on the two most resistant RI tested, RI-4 and RI-272. Of the PI lines, PI-229358 usually produced the lowest weight larvae, similar to larval weight produced on RI-4 and RI-272. In these, as in previous tests, Minsoy and Noir 1 average trait values were intermediate to the resistant and susceptible RI for all traits.

Soybean looper bioassays. When the extremely resistant or susceptible RI phenotypes were tested on soybean looper in a set of eight different bioassays, the trends were generally similar to those found in tests with H. zea (Table 4). Larval weight was highest and larvae developed fastest on a susceptible line, RI-16, than on any other line tested. Resistant lines (both RI-272 and RI-4 and two of the PIs) produced significantly smaller and slower developing larvae than the RI parents or two susceptible RI lines, RI-16 and RI-222. Once more, the ‘Minsoy’ and ‘Noir 1’ parent trait values were intermediate to the resistant and susceptible RI lines. Percent survival on Noir 1 was not significantly different from that of the susceptible RI-16.

Infestations by other arthropods. On different occasions, two other arthropod species infested sets of plants planned for bioassays. These infested plants were not used for bioassays but were censussed for infestation levels after we allowed the pests to persist for another week. The greenhouse whitefly, Trialeurodes vaporariorum (Westwood) (Homoptera: Aleyrodidae), infestation occurred on a set of 16 different RI lines and the two parents, five pots of each. The resistant RI-4 standard was significantly less infested than the susceptible standard, RI-55 in % infested leaves (13 vs 29%), adults per leaf (0.1 vs 0.8) and immatures per leaf (0.38 vs 1.8). Noir 1 had the highest percentage of infested leaves, but had intermediate densities of both adults and immatures, as did Minsoy. A second species, the two spotted spider mite, Tetranychus urticae Koch (Acari: Tetranychidae), infested a set of plants which included only RI-272 (resistant), RI-16 (susceptible) and the resistant PI-227687, nine pots of each line. RI-16 had the highest percentage of infested leaves (50%) and infested leaflets (37%), while RI-272 and the PI line had significantly lower % infested leaves (24% and 14%, respectively) and leaflets (9 and 6%, respectively). Of the infested leaves, 95% of RI-16 leaflets were infested, while levels for both the resistant lines were significantly lower, 43% and 38%. The results from the insect infestations along with the other bioassays demonstrate that the plant breeding methods of the invention are applicable to affecting interactions with diverse pests.

QTL associated with resistance/susceptibility. Several independent QTLs were associated with the traits measured (Table 5). Two significant QTLs were associated with larval weight. One occurs on Utah linkage group U2, linked to the marker T183. Another, bracketed by the linked markers, BL019A and Satt302, occurs on U10. This locus on U10 (linked to Satt302) also affects pupal weight as does another distinct and distant QTL on U10 linked to marker A089. This second QTL has no effect on larval weight.

QTLs affecting larval survival to the pupal stage were linked to markers on U8 and U10 (Table 5). The same QTLs affected survival to the prepupal period. Larval developmental rate QTLs were linked to markers on U2 and U10, and nutritional measure QTLs to markers on U2 and U6. It is apparent from the data in Table 5 that some QTLs influence several traits, e.g., the QTL linked to T183 regulates larval weight, developmental rates and digestive efficiency, and QTL linked to BL019A exerts an effect on larval weight, developmental rate and survival. In contrast, QTLs on U8 and U6 uniquely affect either survival or digestive efficiency measures, respectively.

The results with H. zea indicate that recombinants from a cross of genetically different, non-resistant, soybean parents produce segregants with a larger range of defensive effects than the parents. Further evidence was obtained for the generality of the resistance of these RI lines with respect to other arthropod species. Firstly, selected resistant RI phenotypes, based on H. zea bioassays, were also resistant to a second lepidopteran, the soybean looper, P. includens. Secondly, similar patterns of resistance/susceptibility were observed with natural infestations of the greenhouse whitefly and the two spotted spider mite. Finally, resistance levels of several RI were similar to those of the USDA PI foreign soybean accessions, PI-171451, PI-227687, and PI-229358, known to be resistant to lepidopteran and coleopteran pests (Khush, G. S. and Brar, D. S. (1991) Advances in Agronomy 45:223-274). Thus the breeding methods of the invention can produce progeny plant lines with superior interactions with interacting organisms. Quantitative trait loci associated with affecting an interaction between a plant and an interacting organism were also identified. QTLs identified through these breeding methods can be bred into other plant lines to produce progeny with a desired complement of quantitative traits of agronomic importance.

Early instar mortality in our studies was lower than mortality in some of the other studies of resistant soybeans (Smith, C. M. (1985) Insect Science and its application 6:243-248); however, results of Hatchett, J. H. et al. (1976) Crop Science 16:277-280, Lambert, L. and Kilen, T. C., (1984) Crop Science 24:163-164, and Beach, R. M. and Todd, J. W. (1988) J. Economic Entomology, suggest that early larval mortality is quite variable from test to test. Because specific test and measurement conditions vary between these studies, it is difficult to make direct comparisons. For example, in the experiments reported herein, extra precautions were taken to minimize neonate mortality due to handling and transfer. Results of different studies do agree, however, that lepidopteran larvae are most affected in later instars as the highest mortality occurs just prior to pupation.

The insect response to different RI segregants is quantitative. It seems likely that the plant defensive traits responsible for these differences also are quantitative. Not unexpectedly, several plant genes regulated these traits. Thus, several putative QTLs found on different linkage groups affect larval growth and development, pupal weight and survival. Some QTLs are unique and affect only one trait, whereas others, such as T183 on U2 and BL019A on U10, affect several traits. Combinations of both Noir 1 and Minsoy parental alleles contribute to the resistance, but most of the resistant alleles are associated with the Minsoy allele (Table 5). Indeed, the average trait values for each parent suggest that Noir 1 is a better substrate than Minsoy. The effects of the QTL linked to T183 in an independent RI population were confirmed in a cross between Minsoy and an elite cultivar, Archer (Terry et al. (2000) Crop Science 40 (2): 375-382). Moreover, a detailed QTL analysis suggests that other resistant Noir 1 alleles also contribute to the increased difference between the resistant and susceptible phenotypes (Terry et al., (2000) supra).

The patterns of QTL associations also can explain the patterns of correlations between traits. Larval weight and developmental rates are highly correlated, and both of the larval weight QTLs also affect developmental rates. Correlations among other traits are not as strong as that between larval weight and developmental rates. This also is reflected in weaker QTL associations. Thus, pupal weight, % survival and the nutritional indices all have at least one unique QTL for each trait.

Various phenotypes of hybrid plant swarms were proposed or observed as hosts for herbivores (i.e., increased resistance, decreased resistance, intermediate effects, or resistance like one or the other parent). All of these phenotypes were observed when H. zea was reared on the RI population. Many of the RI phenotypes were quantitatively similar to those of their parents (FIG. 2), but some were more extreme, extending the phenotypic range of the RI lines far beyond the parental values. Resistant alleles for different genes can be inherited from each parent resulting in a combined cumulative effect for more resistance than either parent alone. More extreme susceptibility can arise in the same way.

The variety of responses to the different RI lines contrasts with studies of herbivore responses to hybrid plants, where only one of these several alternatives could be observed (Strauss, S. (1994) Trends in Ecology and Evolution 9:209-214). In natural hybrid zones, many factors may contribute to the variation in hybrid resistance: those that determine which hybrids succeed in an environment (sterility, unequal ploidy, genetic drift) (Arnold, M. J. (1992) Ann. Rev. Ecology and Systematics 23:237-261); the degree of backcrossing with or introgression by one or the other parent's genes (Keim, P. M., et al. (1989) Genetics 123:557-565; Paige, K. N. and Capman, W. C. (1993) Evolution 47:36-45; Strauss, S. (1994) Trends in Ecology and Evolution 9:209-214; Whitham, T. G. et al. (1994) Oecologia 97:481-490); the genetic nature of the resistance (dominance, monogenic versus polygenic traits, epistatic effects) (Fritz, R. S. et al. (1994) Oecologia 97:106-117); the insect species or race present in the hybrid zone locale (Karban, R. (1989) Nature 340:60-61; Fritz et al., 1994, supra; Strauss, S. Y. and Karban, R. (1994) Evolution 48:454-464; Messina, F. J. et al. (1996) Oecologia 107:513-521; Orians, C. M. and Floyd, T. (1997) Oecologia 109:407-413); environmentally induced effects and plant phenology (Kearsley, M. J. C. and Whitham, T. (1989) Ecology 70:422-434; Floate, K. D. et al. (1993) Ecology 74:2056-2065; and tritrophic interactions (Gaylord, E. S. et al., (1996) Oecologia 105:336-342). Detailed studies have only begun to elucidate the extent and effects of these factors. The invention described herein shows that a wide range of defensive plant phenotypes can result from introgression of resistance when many genes are involved, and similar effects can be observed in hybrids and their segregating or backcrossing progeny. Selection can then operate to eliminate all but one phenotype.

Complex, polygenic resistance probably evolved during the lengthy co-existence of plants and their enemies, including insect predators. Although many of these resistance alleles may have been lost or sequestered during domestication, naturally occurring resistance has been found in locally adapted cultivars of a crop species; in foreign accessions within a cultivated species; and in natural hybrids between crop species and wild relatives. These sources have been used to find resistant germplasm of corn, tomatoes, potatoes and soybeans (Painter, R. H., Insect Resistance to Crop Plants, University of Kansas Press, Lawrence, Kans., 521 pp. (1951); Smith, C. M., Plant Resistance to Insects: A Fundamental Approach (1989) John Wiley and Sons, New York, 286 pp.; Stoner, K. A. (1996) Biological Agriculture and Horticulture 13:7-38). The results described herein suggest that it also may be useful to cross genetically different cultivars (even relatively susceptible ones) to produce recombinants with a wide range of defensive phenotypes against insect herbivores. This approach offers advantages if it is accompanied by genetic mapping data. QTLs of traits can be linked to markers on the genetic map, and these markers can serve as tags for marker-assisted selection in breeding programs to determine whether segregants have the desired QTLs.

It was surprisingly discovered by the inventors that soybean recombinants derived from the cross of relatively susceptible, but unrelated, lines transgressively segregate to produce resistant and susceptible phenotypes. It was also demonstrated that the quantitative nature of these defensive traits are controlled by several genes on different linkage groups and that the resistance and susceptibility of individual RI lines appear to be of a general nature affecting a variety of interacting organisms.

Further studies were conducted with other soybean cultivars to assess the generality of the breeding methods of the invention. In these studies the breeding methods of the invention were analyzed using the Minsoy and Archer cultivars.

The values for the two RI populations reflect the differences in trait values between parents, Archer being a better corn earworm host than Noir 1 (Table 6). In both populations the larval weight segregated in a transgressive manner (P<10⁻⁴) as did the pupal weight in the MN population (P<10⁻⁶). (For example, 26 lines in the MA and 27 lines in the MN were outside the 95% confidence limits set for larval weight by the values of the parental lines.)

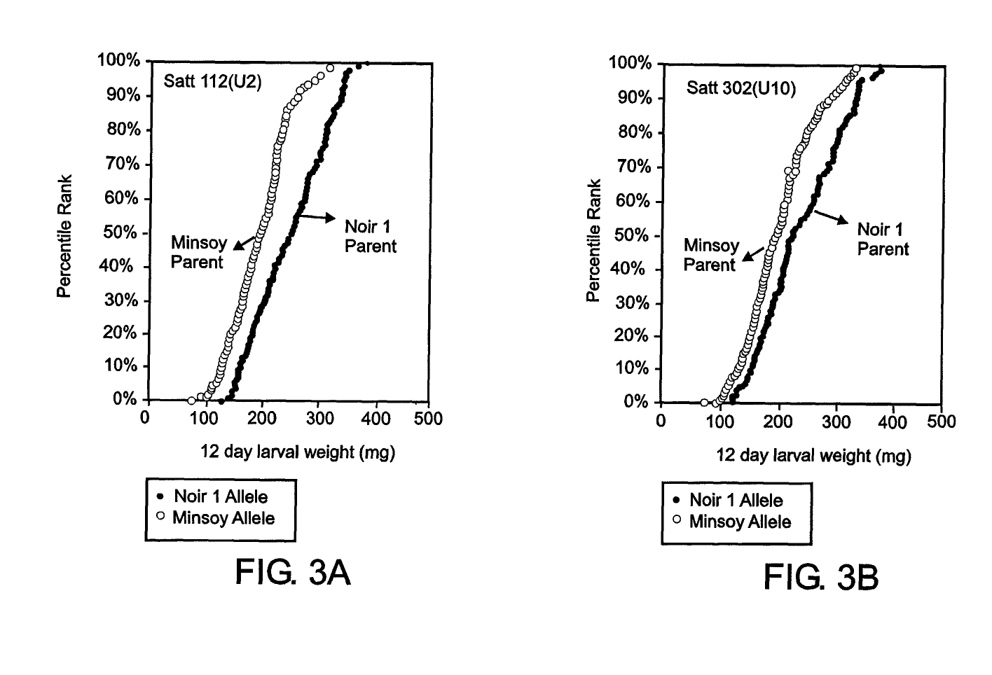

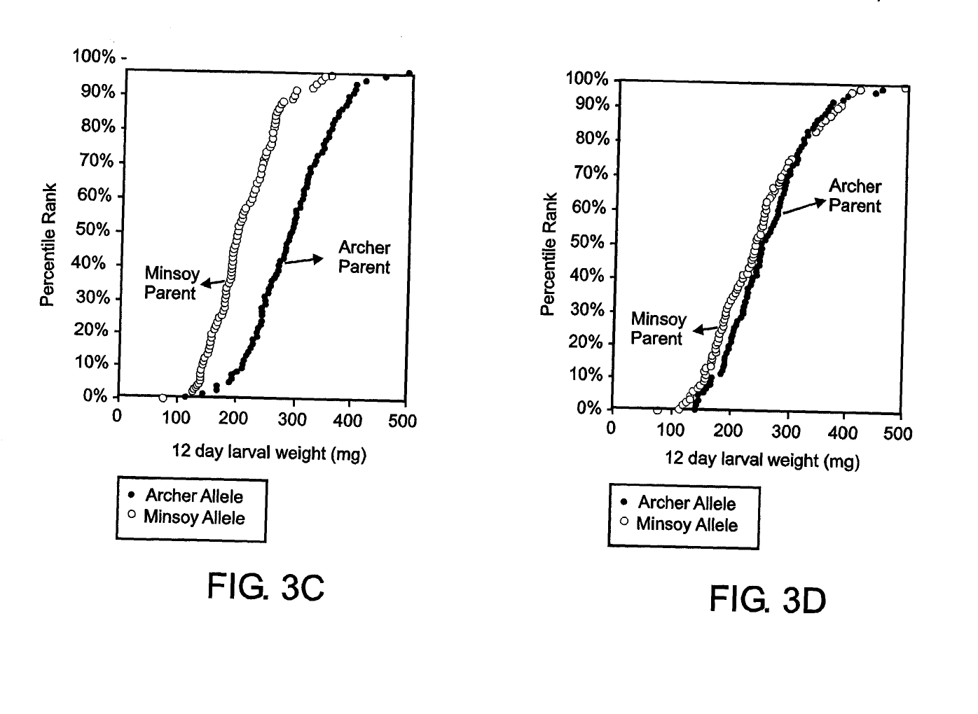

Previous results had indicated that significant QTLs detected in the MN population were associated primarily with linkage groups (LGs) U2 and U10 (Terry et al., 1999, supra) of the 20 LGs of the composite soybean map (Cregan et al., 1999, supra). The cumulative distributions of larval weights associated with parental alleles of loci on these linkage groups show the degree of difference between larval weights associated with each allele (FIG. 3). Evidence for a QTL associated with LG U2 is found in both populations (FIGS. 3, A and C) confirming this QTL. In contrast, analysis of the data identified a QTL in LG U10 in the MN population (FIG. 3, B) but not in the MA population (FIG. 3, D).

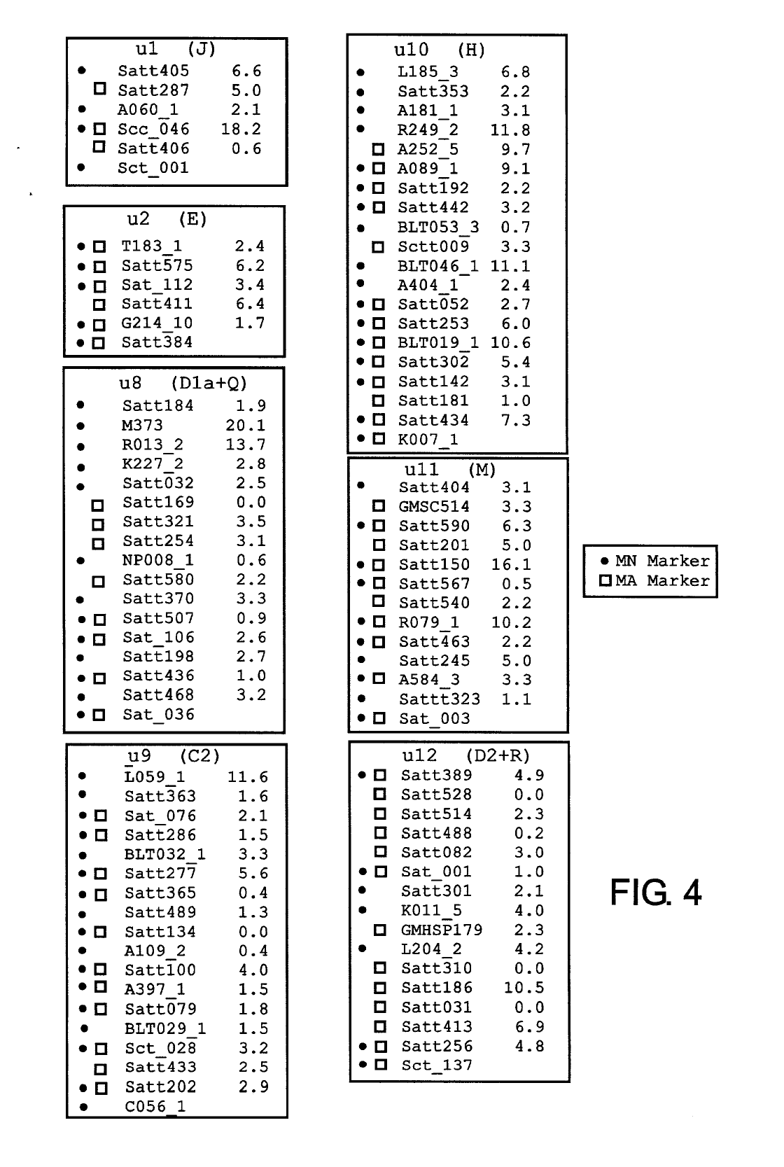

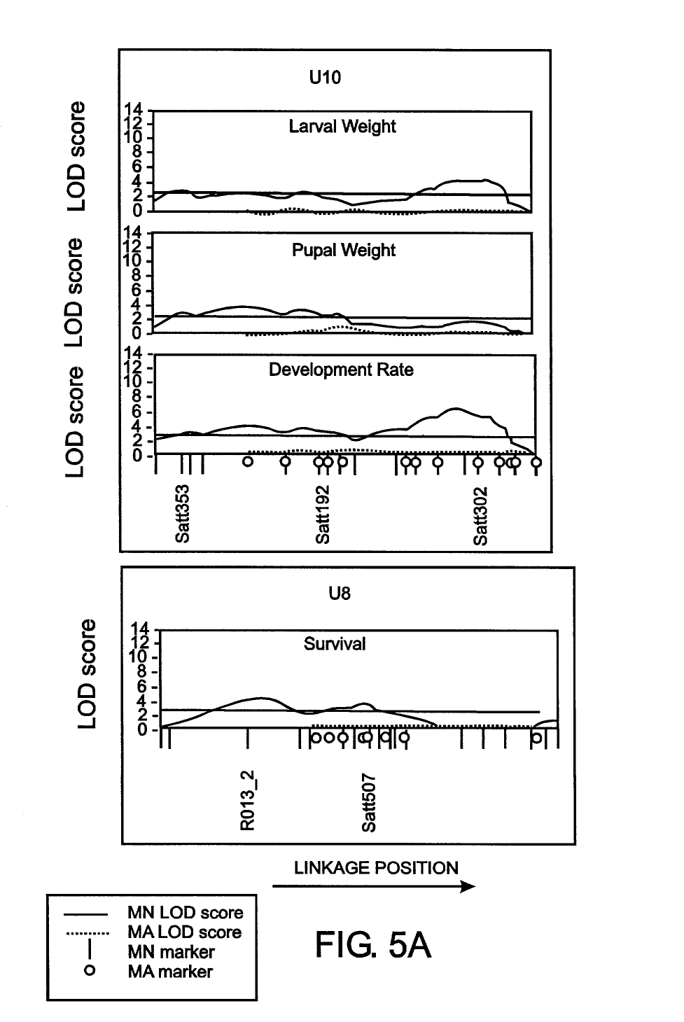

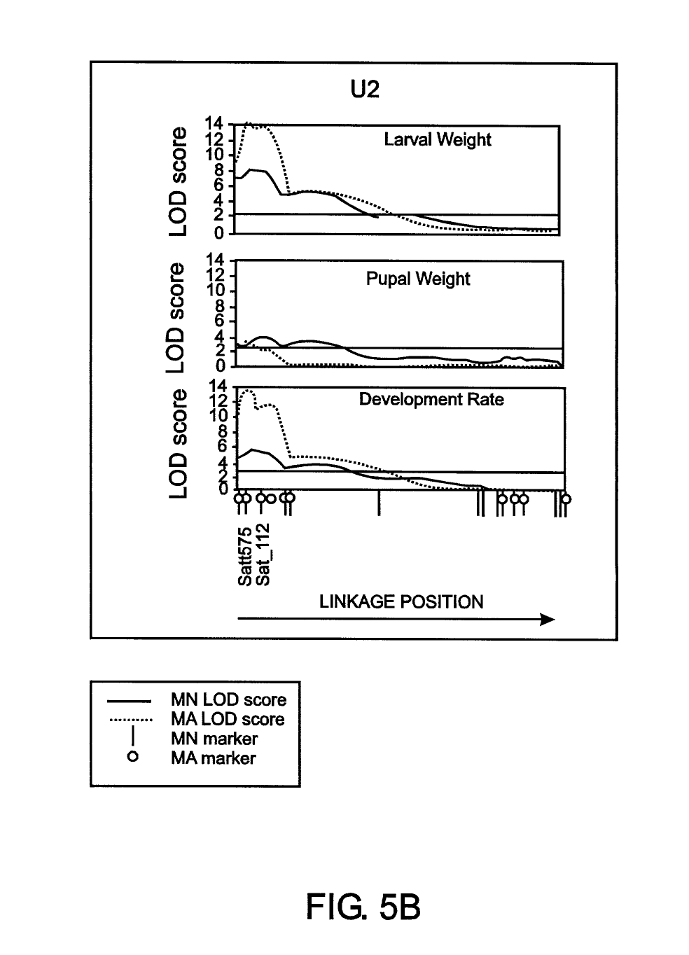

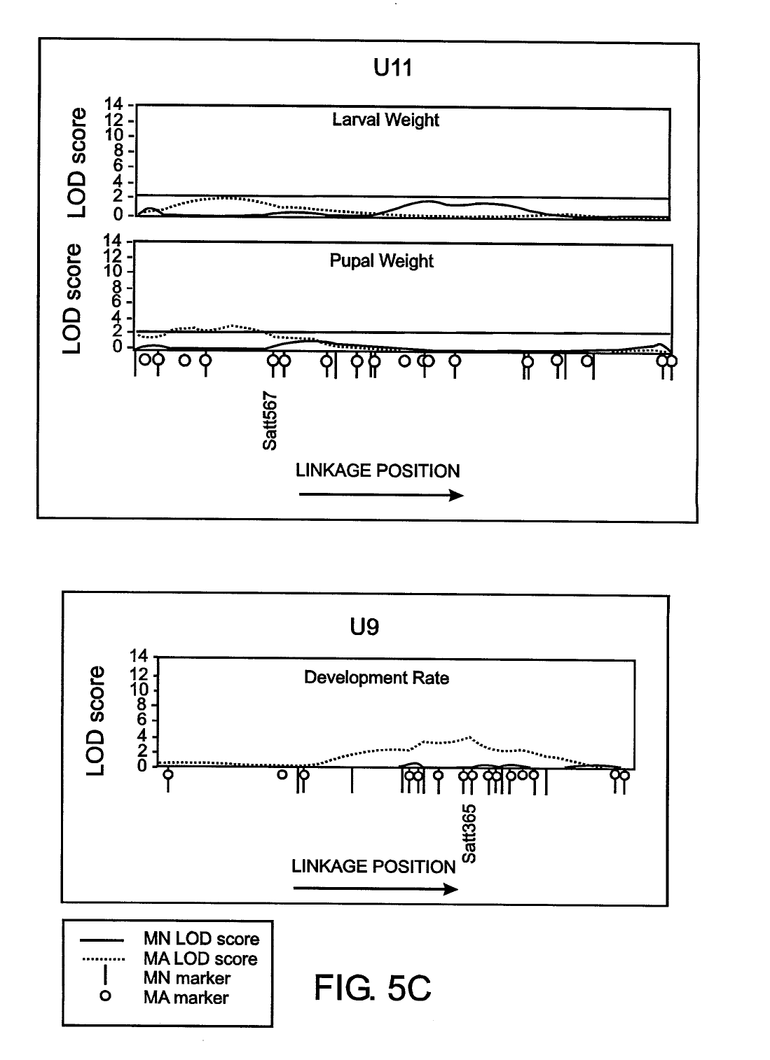

Not all of the molecular markers segregated in both the MN and MA populations (FIG. 4). Where markers segregated, it was possible to test for segregation of linked QTLs. In LG U2 markers segregated in both populations and QTLs also were shown to segregate in both populations (FIGS. 3 and 5, Table 7). In LG U10, Satt302 segregated in both populations (FIG. 4), but a QTL linked to this marker only segregated in the MN population (FIGS. 3 and 5, Table 7). Several other QTLs were identified in either the MN or MA populations (FIG. 5, Table 7). Many of these were linked to markers that segregated in both populations. In these cases, it was possible to determine that a QTL segregating in one population was not segregating in the other. Thus a QTL for survival identified on LG U8 in the MN population (linked to Satt507) did not segregate in the MA population (FIG. 5, Table 7). Similarly, a QTL associated with Satt365 in LG U9 in the MA population did not segregate in the MN population, nor did QTLs found in LG U11 linked to Satt567. In other cases, QTLs linked to segregating markers in one population could not be tested for linkage in the other, because segregating markers were not available. This was the case for QTLs linked to R013_{—}2 in LG U8 and to Satt353 in LG U10 (FIG. 5, Table 7).

Most of the resistance alleles are derived from the Minsoy parent (Table 7). These include resistance alleles associated with LGs U2 and U10, as well as one for pupal weight on LG U11. The tests lack enough sensitivity to determine conclusively if the QTLs on LG U10 are separate, but based on the point mapping P-values of individual markers (Table 7) three were chosen to fit QTLs to the region. One of these (linked to Satt353) primarily affects larval weight and 42.7 cM distance away is another (linked to Satt192) that affects larval weight, pupal weight, and development rate. A third QTL (linked to Satt302) primarily affects larval weight and development rate, but not pupal weight.

The most important QTL is found on LG U2. It is observed in both populations, it accounts for the largest fraction of phenotypic variation and affects three traits: larval weight, pupal weight, and development rate (Table 7). This QTL, linked to Satt575 and Sat_{—}112, was first detected in the MN RI population. It was confirmed in a different genetic background (the MA RI population) derived from the elite soybean cultivar, Archer. Because Minsoy is a somewhat exotic P.I., it seems unlikely that this resistance allele is present in most of the elite germplasm currently in use and because the allele remains active when crossed with Archer, it is likely that the resistance will not be lost if introgressed into other elite germplasm. These results have a direct application in developing insect resistant germplasm. QTLs for many agronomic traits have been identified and mapped in these RI soybean populations (Mansur et al., 1996, supra; Orf et al., 1999, supra). As yet, no important agronomic QTL has been found that is linked to the major resistance QTL on LG U2. This information is useful for planning breeding strategies to minimize the effects on important agricultural traits while gaining resistance. It also suggests that the direct cost of resistance to the plant in terms of yield, or other traits, may be minor.

Some minor resistance alleles, derived from the Noir 1 parent, are found on LGs U1, U8, and U12 (Table 2). The most important of these are two for survival located on LG U8 and separated by more than 25 cM from each other (FIG. 2, Table 2). No resistance alleles were detected from the Archer parent. However, the fact that many MA RI segregants are more resistant than the Minsoy parent (FIG. 1) suggests that additional resistance genes derived from Archer may exist.

Quantitative traits are traditionally viewed as being controlled by a large number of loci each with a small effect (r²≦5%) that in the aggregate affect the phenotype (Tanksley, S. D. (1993) Ann. Rev. Genet. 27:205-233). However, many of the QTLs directly associated with the variation in insect growth and development are not minor (Table 7). In the MN RI population, the largest of these, linked to SAT_{—}112 on LG U2, accounts for 17% of the variation in larval weight and 12% of the developmental rate. In the MA RI population the QTL on LG U2 accounts for 28 and 29% of the variation in the developmental rate and the larval weight. Moreover, in the MN population there were several other QTL associated with resistance traits, each accounting for more than 5% of the total variation. However, where multiple QTL are associated with a trait, the sum of the individual QTL effects may not be additive. The VQTL (R²), determined through multiple regression models, adjusts for multicolinearity among QTLs and gives a measure of the total percent of the variation explained by all the QTL. In most traits (Table 3), the total VQTL is similar to the sum of the individual QTL effects in Table 2.

The broad sense heritability values are moderate (range of 42-65) (Table 3) and suggest that the expected gain due to selection would be moderate. Except for pupal weight, the heritabilities between the populations are similar. In the MA population most of the variation in larval and development rate is associated with the LG U2 locus, whereas several loci contribute to the variation in the MN population; but the U2 QTL is the only major locus (r²>10%, Table 2 and 3) in either population. These data further support the conclusion that the resistance associated with the Minsoy allele of QTL on LG U2 is of primary importance. In addition, the other MN QTL alleles may prove useful to breeders when placed in the context of elite genetic backgrounds other than Archer, as may the survival QTL alleles (LG U8) derived from the Noir 1 parent.

Resistance modalities to herbivores are broadly grouped into one of the following categories: antibiosis; antixenosis; or tolerance of the pest. The bioassays tested for antibiosis factors, demonstrating that the resistance gene on LG U2 affected both larval weight gain and development rates (Table 2, FIGS. 1 and 3). The breeding methods of the invention can be used to breed plants with enhanced resistance modalities towards interacting pest organisms, and particularly insect pests. Such enhancements of resistance modalities can be through antibiosis, antixenosis, or tolerance of pest type mechanisms.

Many of the procedures useful for practicing the present invention, whether or not described herein in detail, are well known to those skilled in the art of plant molecular biology. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; and Setlow and Hollaender (1979) Genetic Engineering: Principles and Methods, Vols. 1-4, Plenum Press, New York, Kaufman (1987) in Genetic Engineering Principles and Methods, J. K. Setlow, ed., Plenum Press, NY, pp. 155-198; Fitchen et al. (1993) Annu. Rev. Microbiol. 47:739-764; Tolstoshev et al. (1993) in Genomic Research in Molecular Medicine and Virology, Academic Press. Abbreviations and nomenclature, where employed, are deemed standard in the field and commonly used in professional journals such as those cited herein.

### EXAMPLE 1

It is demonstrated, herein, that by crossing two genetically distinct parental plant varietals a RI population of soybeans can be created that contains a diversity of defensive phenotypes that affect several different herbivorous arthropods, and that these defenses measured across the RI population appear to be due to multiple gene effects.

The RI soybean population and genetic marker data. The specific methods for developing this RI soybean population have been described previously (Mansur, L. M. and Orf, J. H., (1995) Crop Science 35:422-425; Mansur, L. M. et al. (1996) Crop Science 36:1327-1336). In brief, more than 250 RI lines were derived using a single seed descent from F2 segregants produced on reciprocal crosses of Noir 1 (PI 290136) from Hungary and Minsoy (PI 27890) from China. The seed used in the following experiments were from F13 or F14 generation seed stock.

Genetic markers for mapping the RI consist of restriction fragment length polymorphisms (RFLPs), simple sequence repeat markers (SSRs) and a few classical morphological markers. Procedures for developing these markers are detailed elsewhere (Apuya, N. R. et al. (1988) Theoretical and Applied Genetics 75:889-901; Keim, P. and Shoemaker, R. C. (1988) Soybean Genetics Newsletter 15:147-148; Akkaya, M. S. et al. (1992) Genetics 132:1131-1139; Lark, K. G. et al. (1993) Theoretical and Applied Genetics 86:901-906; Cregan, P. B. et al. (1994) Methods of Molecular and Cellular Biology 5:49-61; Akkaya, M. S. et al. (1995) Crop Science 35:1439-1445; Mansur, L. M. et al. (1996) Crop Science 36:1327-1336). Markers described herein can be obtained from the laboratories of K. G. Lark, University of Utah, Salt Lake City, Utah, R. C. Shoemaker, Iowa State University, Ames, Iowa, P. N. Keim, Northern Arizona University, Flagstaff, Ariz., or from Biogenetic Services, Inc. 2308-6^{th} Street East, Brookings, S.Dak. 57006. Minsoy and Noir 1 were screened for RFLP polymorphisms by hybridization against southern blots of their DNA restriction fragments. Polymorphic RFLP markers were then hybridized against southern blots of restriction fragments of DNA from RI lines. DNA preparations from the RI lines were used to determine the SSR alleles of each SSR marker. These polymorphic SSR loci are presented elsewhere (Mansur et al., 1996, supra; Cregan, P. B. et al. (1999) Crop Science 39:1464-1490) and the corresponding primers are commercially available. The primer sequences for synthesis of SSRs are available on the internet at the “Soybase” website whose address is http://129.186.26.94. A list of SSRs presented at the primer sequence for a desired SSR is revealed by clicking on the desired SSR in the list.

From the genetic data set used (comprised of 246 RFLPs, 338 SSRs and three classical markers), approximately 2700 cM and 22 linkage groups were defined corresponding to the n=20 chromosomes of soybean. Linkage of loci and mapped distances were determined using two programs, Mapmaker 3.0 (Lander, E. S. et al. (1987) Genomics 1:174-181; Lincoln, S. E. and Lander, S. L., Mapmaker/exp 3.0 and Mapmaker/QTL 1.1., (1993), Whitehead Inst. of Medical Research Technical Report, Cambridge, Mass.) and Join-Map (Stam, P. (1993) The Plant Journal 3:739-744) as reported by Cregan et al. (1999), supra.

Herbivore bioassays. The corn earworm, Helicoverpa zea (Boddie), a polyphagous New World foliage and fruit feeding species and a major pest of soybeans (Fitt, G. W. (1989) Annual Review of Entomology 34:17-52; McPherson, R. M. and Moss, T. P. (1989) J. Economic Entomology 82:1767-1772), was used as the primary test herbivore in the study. Eggs were obtained from the USDA-ARS Insect Biology and Population Management Research Laboratory in Tifton, Ga. This colony has been maintained for over 20 years with no infusion of outside individuals (Young, J. R. et al. (1976) USDA-ARS-S 110:4 pp.). From preliminary data on another colony (eggs obtained from North Carolina State University Insect Rearing facility), a susceptible and resistant RI standard was determined. In tests with both colonies, the resistant standard (RI-4) was among the most resistant lines and the susceptible standard (RI-55) was among the most susceptible RI lines. Data from only the GA colony was used in the QTL analysis.

Excised leaf tissue was used in a no-choice test to determine relative effects of each RI line on larval development. Due to time and space constraints, only 18-24 RI lines were tested in a single experiment, and ten larvae were used per line in each experiment, where a single larva was the experimental unit. Over three years, each RI line was tested in a minimum of two different bioassays, and many were tested three or more times.

Neonate larvae were placed on young terminals or expanding trifoliates inside 160 ml plastic rearing cups, at one larva per cup, and were maintained at 27° C. To eliminate mortality due to handling, two larvae were placed per cup in two of ten cups per line initially and then thinned those to one per cup at day three of larval development. Fresh leaves were added at least every 48 h. Data recorded were: larval weight at 8, 10 and 12 days; pupal weight between 24-48 h after pupation; development time to reach prepupal and pupal stage (transformed to rate to linearize the relationship); % survival to the 12^{th} day of larval growth, to the prepupal period and to the pupal stage. Digestive efficiency measures were calculated using gravimetric methods based on dry weight ratios of subsets of leaf tissues and larvae (Waldbauer, G. P. (1968) Advances in Insect Physiology 5:229-288; Kogan, M., In: J. Miller and T. Miller (eds.) (1986) Insect-Plant Interactions, Springer-Verlag, New York, pp. 155-190) during days 8-12. One problem associated with ratio-based nutritional measures is due to the allometric rather than isometric relationship between larval mass and food intake (Raubenheimer & Simpson, 1992; Raubenheimer & Simpson, 1994). The relationship between these parent variables in the data set did not significantly deviate from isometry during the time period when these measurements were taken. Three nutritional measures were used as indicators of the resistance found in these soybean lines: CI, consumption index; ECI, % efficiency of conversion of ingested food into body mass; and ECD, % efficiency of conversion of digested food into body mass.

For each test, at least six plants of each RI line were grown in a quarantine greenhouse to minimize exposure to other insects. Supplemental artificial lighting consisted of combinations of 75 W fluorescent and 400 W high pressure sodium lamps at L18:D6 to maintain plants in vegetative stages. Ten days to two weeks prior to testing, plants were moved to a larger greenhouse with combinations of 1000 W high pressure mercury and high pressure sodium lamps. Plants were in pre-bloom to early blooming stages (V6-V10 to R1-R2, Fehr, W. R. and Caviness, C. E. (1977) Iowa Cooperative Extension Service Special Report :80) at test initiation. Greenhouse temperature was maintained at an average of 25° C.

The effects of selected susceptible and resistant RI lines were compared with those of the known resistant USDA PI lines in bioassays similar to those described above. In addition, the effects of several extreme RI phenotypes were examined on a second insect pest, the soybean looper, Pseudoplusia includens (Walker) (Lepidoptera: Noctuidae), an oligophagous foliage feeding caterpillar. Eggs were obtained from the Insect Rearing Laboratory at USDA-ARS, Stoneville Miss.

Statistical analyses. Analysis of variance, GLM, (SAS Institute Inc., 1988) was used to test for the effects of RI lines on each variable measured. Linear contrasts were used to make specific comparisons (e.g., each RI with the standard RI). Correlations among traits were calculated by Pearson Product moment (SAS Institute Inc., 1988).

Identification of resistance/susceptibility plant QTLs. Standardized trait values were used to identify plant QTLs. Although plant stage and bioassay conditions were similar across all experiments, average larval weights at 12 days and developmental rates varied from test to test, possibly due to variations in plant growth and greenhouse conditions across seasons. Therefore, trait values (except pupal weight) within each test were standardized in two different ways: i) to an average value over all tests; and ii) to the susceptible standard average across all tests. These adjusted larval weight characters are highly correlated with each other (r=0.93, P<0.0001) and with the unadjusted larval weight data (r=0.81 to 0.83, P<0.0001). Similarly, developmental rates and nutritional measures adjusted and raw values are highly correlated (r=0.64 to 0.95).

The Minsoy X Noir 1 RI population was mapped (Lark et al., 1993, supra; Mansur et al., 1996, supra; Cregan et al., 1999, supra; Orf et al., 1999, supra) using the molecular markers described above. The allele at each marker from this mapping data was determined for each RI line. Because each RI line is homozygous, the genotype for each marker was assigned as a single allele, ‘A’ for Noir 1 and ‘B’ for the Minsoy allele. These marker data were used in conjunction with the trait values to identify QTLs.

Several computer programs were used to test for QTL linkage with larval growth traits, as indicated by significant associations of traits with RFLP or SSR markers. Both least squares (SAS Institute Inc., 1988) and Maximum Likelihood (ML) models (Lark, K. G. et al. (1995) Proc. Natl. Acad. Sci. 92:4656-4660; Chase, K. et al. (1997) Theoretical and Applied Genetics 94:724-730) were used to determine candidate QTL associated with resistance traits in the RI population. The SAS/IML program (SAS Institute Inc., 1989) was used to more efficiently analyze the entire marker data base for significant QTL with GLM. This program is based on a model by E. S. Edgington, “Randomization Tests,” ([1980] Marcel Decker, Inc., New York, 287 pp.), to test the effect of each locus and to create an output data set consisting of only significant QTLs for each trait at a threshold, P<0.001 (Lander, E. S. and Botstein, D. (1989) Genetics 121:185-199). The average trait value and the number of RI associated with each genotype, the P value for the GLM and the percent of the variation explained (R²) by the model were also determined. The QTLs were further tested for significance by the sequential Bonferroni test, Dunn-Sidak method based on 500 comparisons at an experimentwise error rate of 0.05, (Sokal, R. R. and Rohlf, F. J., “Biometry: The principles and practice of statistics in biological research,” (1995) 3d ed., W. H. Freeman and Company, New York, 887 pp.).

Direct effects of each marker were tested independently using EPISTAT (Chase et al., 1997 supra), a program that uses ML models to determine the log likelihood ratio (LLR) of the effect associated with a marker (QTL) as opposed to its occurrence by random chance (null model). The null model was rejected at a threshold LLR score of approximately 5.0 (˜P<=0.001, determined by Monte Carlo simulations of random selection of sub-populations of the data set). Monte Carlo simulations (1 million runs each) were used to determine the probability of exceeding the LLR. The QTL results between the GLM and ML methods agreed completely.

Significant loci on the same linkage group were considered as separate QTL if they were >50 cM apart, if markers between them had significantly lower LLR or probability values (Mansur et al., 1996, supra), or if there was a reversal in the resistant allele. These statistical tests were completed on 240 RI lines. Each line not included was eliminated because of its high level of heterozygosity or its near genetic identity to other RI that were analyzed.

TABLE 1
Growth measurements of Helicoverpa zea, when reared on the indicated soybean cultivars, including the parents,
Noir 1 and Minsoy, resistant RI lines 4, 67 and 272 and susceptible
RI lines 16, 55, and 327, the means and ranges of all RI lines

| | Weight (mg) | | | | | | Rate of larval¹ | | Nutritional² | | | % Survival³ | |
|---|---|---|---|---|---|---|---|---|---|---|---|---|---|
| | 12 day old larva | | | Pupa | | | development to | | efficiency index | | | to | |
| Cultivar | All⁴ | Female⁴ | Male⁴ | All | Female | Male | Prepupa | Pupa | CI | % ECI | % ECD | 12 days | Pupa |
| RI-4 | 147 | 179 | 174 | 241 | 242 | 242 | 5.4 | 4.9 | 5.6 | 6.0 | 10.0 | 92 | 59 |
| RI-16 | 418 | 415 | 417 | 279 | 281 | 276 | 7.3 | 6.3 | 3.4 | 10.6 | 20.3 | 98 | 96 |
| RI-55 | 360 | 359 | 378 | 268 | 268 | 271 | 7.3 | 6.2 | 3.6 | 9.0 | 18.6 | 97 | 89 |
| RI-67 | 142 | 183 | 163 | 233 | 235 | 237 | 5.4 | 4.8 | 5.3 | 6.5 | 13.2 | 97 | 66 |
| RI-272 | 85 | 124 | 115 | 185 | 211 | 181 | 4.8 | 4.4 | 6.4 | 4.9 | 7.5 | 92 | 30 |
| RI-327 | 342 | 368 | 326 | 308 | 296 | 318 | 6.8 | 5.8 | 3.2 | 9.9 | 18.4 | 92 | 87 |
| Noir1 | 268 | 258 | 269 | 261 | 247 | 236 | 5.9 | 5.2 | 4.9 | 9.2 | 19.1 | 100 | 96 |
| Minsoy | 229 | 233 | 224 | 251 | 231 | 240 | 5.8 | 5.2 | 4.4 | 10.3 | 16.6 | 100 | 82 |
| RI avg. | 222 | 248 | 239 | 243 | 240 | 248 | 5.9 | 5.3 | 4.2 | 8.2 | 14.6 | 94 | 74 |
| min | 85 | 76 | 85 | 185 | 183 | 180 | 4.8 | 4.4 | 2.0 | 4.8 | 7.1 | 78 | 26 |
| max | 418 | 415 | 417 | 308 | 330 | 320 | 7.3 | 6.3 | 6.6 | 17.9 | 28.0 | 100 | 100 |

¹Rate of larval development to indicated stage = 1/(number of days to become prepupa or pupa) × 100
²Cl₂ consumption index = 100 × (mg dry wt. of food eaten)/(avg. larval dry wt., days 8-12) ECI, % efficiency of conversion of ingested food into body mass = 100 × (mg. dry wt. gain of larvae)/(mg dry wt. of ingested food) ECD = % efficiency of conversion of digested food into body mass = 100 × (mg. dry wt. gain of larvae)/(dry wt. ingested food − dry wt. of frass)
³% survival to 12^{th} day of larval growth and % survival to the pupal stage (cumulative)
⁴For RI producing a high mortality, the male and female larval weights may be much higher than the average of all larvae because sexed larval weights are based on only those that survive to pupal stage, when sexes are first visually identified.

TABLE 2
Correlation coefficients among Helicoverpa zea growth traits and survival when
reared on Minsoy X Noir1 recombinant inbred lines, data averaged across all tests¹

| | | | | | Nutrition | | |
|---|---|---|---|---|---|---|---|
| | | Larval | Pupal | Devel.³ | index⁴ | | % Survival to |
| Trait² | | Wt. | Wt. | rate | CI | ECD | 12 day larva |
| Larval weight at 12 days | 1.00 | | | | | | |
| Pupal weight | 0.35 | 1.00 | | | | | |
| Larval developmental rate³ | | 0.80 | 0.39 | 1.00 | | | |
| Nutritional index, CI⁴ | | −0.61 | −0.19 | −0.47 | 1.00 | | |
| Nutritional index, ECD⁴ | 0.55 | 0.23 | 0.40 | −0.78 | 1.00 | | |
| % Survival to 12 day larva | | 0.24 | 0.17 | 0.18 | −0.16 | 0.16 | 1.00 |
| % Survival to pupal stage | | 0.62 | 0.37 | 0.45 | −0.34 | 0.35 | 0.44 |

¹Correlation coefficients, r, are significant (P < 0.01), Pearson product moment, SAS 1988, n = 237-240.
²Only representatives of each trait type are presented; traits not shown are highly correlated with similar trait types (r > 0.78)
³Devel. Rate, Larval developmental rate to the pupal stage
⁴Nutritional indices, CI, Consumption index; and ECD, % efficiency of conversion of digested food; see Table 1 for full definitions

TABLE 3
Growth measurements of Helicoverpa zea when reared on the
indicated soybean cultivars, comprising the Recombinant Inbred (RI)
parents, Noir 1 and Minsoy, resistant RI lines 4, 67 and 272
and susceptible RI line, RI-16, and resistant USDA foreign
accessions (P1)

| | | Rate of larval | | Nutritional | | | | |
|---|---|---|---|---|---|---|---|---|
| | Weight (mg)¹ | development to^{1,2} | | index^{1,3} | | % Survival to^{1,4} | | |
| Cultivar | 12 day larva | Pupa | Prepupa | Pupa | % ECI | % ECD | 12 days | Pupa |
| RI-4 | 95d | 211c | 4.96c | 4.63c | 5.3bc | 7.9c | 90 | 55 |
| RI-16 | 380a | 285a | 6.26a | 5.67a | 10.9a | 22.5a | 97 | 88 |
| RI-67 | 151c | 238b | 5.38b | 5.07bc | 5.8bc | 10.1bc | 92 | 42 |
| RI-272 | 87d | 205c | 5.5b | 5.15bc | 4.9bc | 7.8c | 90 | 45 |
| Noir 1 | 282b | 230b | 5.84ab | 5.34ab | 9.3a | 16.5ab | 97 | 95 |
| Minsoy | 242b | 261ab | 5.9ab | 5.4ab | 9.6a | 16.5ab | 100 | 87 |
| PI-171451 | 148c | 237b | 5.2b | 4.8c | 4.0c | 5.1c | 90 | 50 |
| PI-227687 | 215bc | 216c | 5.6b | 5.1bc | 7.5b | 14.2b | 92 | 55 |
| PI-229358 | 109d | 227bc | 5.2b | 4.8c | 4.1c | 6.9c | 80 | 40 |

¹The means within columns followed by the same letter are not significantly different by linear contrast, α = 0.01, GLM, (SAS, 1990) of square root transformed data, average of six tests
²Rate of larval development, = 1/(number of days to become a prepupa or pupa) × 100
³Nutritional efficiency indices, days 8-12; ECI, % Efficiency of conversion of ingested food; ECD, % Efficiency of conversion of digested food into body mass; see Table 1 for full definitions
⁴Because variation in mortality across tests was very high, no statistical comparisons were made

TABLE 4
Growth measurements of soybean looper, Pseudoplusia includens,
when reared on the indicated cultivar of soybean¹

| | | | | | Larval develop- | | | | |
|---|---|---|---|---|---|---|---|---|---|
| | Larval weight (mg) at | | | Pupal weight | mental rate³ | | % Survival | | |
| Cultivar² | 8 days | 10 days | 12 days | (mg) | Prepupa | Pupa | 12 days | Prepupa | Pupa |
| RI-4 | 12.6 c | 44.1 c | 98.6 c | 142.3 c | 7.01 c | 6.63 c | 92 | 86 | 82 b |
| RI-16 | 31.4 a | 103.0 a | 186.9 a | 174.2 ab | 8.74 a | 7.96 a | 100 | 100 | 98 a |
| RI-222 | 27.3 ab | 82.3 b | 169.6 a | 168.5 abc | 8.07 b | 7.45 b | 98 | 98 | 94 a |
| RI-272 | 17.2 c | 41.3 c | 95.0 c | 152.7 c | 7.09 c | 6.53 c | 76 | 62 | 59 c |
| Minsoy | 23.3 b | 70.3 b | 139.7 b | 150.7 c | 7.74 b | 7.05 b | 85 | 81 | 78 b |
| Noir 1 | 29.7 a | 81.0 b | 134.2 b | 160.6 bc | 7.99 b | 7.36 b | 95 | 94 | 94 a |
| PI-171451 | 14.3 c | 48.2 c | 118.1 bc | 149.2 c | 6.85 c | 6.37 c | 92 | 83 | 63 bc |
| PI-227687 | 16.0 c | 37.0 c | 113.3 bc | 183.0 a | 6.93 c | 6.46 c | 100 | 84 | 83 b |
| PI-229358 | 15.9 c | 39.9 c | 101.3 c | 149.6 c | 6.55 c | 6.17 c | 84 | 84 | 82 b |

¹Values in each column of data followed by the same letter are not significantly different, linear contrasts, α = 0.05 GLM (SAS, 1990) based on square root transformed data or arcsin square root transformed proportion data, over five tests.
²RI, Recombinant Inbreds (RI) from the cross of Noir 1 and Minsoy parents, RI-16, RI-222 (susceptible); RI-272 and RI-4 (resistant); PI, resistant USDA foreign accession.
³Larval developmental rate, 1/(number of days to become prepupa or pupa) × 100

TABLE 5
Quantitative trait loci (QTL) associated with indicated Helicoverpa zea growth trait
(A = Noir 1 allele, B = Minsoy allele)

| | | Linkage | Avg. of genotype | | Difference | | | |
|---|---|---|---|---|---|---|---|---|
| Trait/stage | QTL¹ | group² | A | B | (A − B) | R² (%) | LLR³ | P, LLR³ |

Weight (mg)⁴

| | | | | | | | | |
|---|---|---|---|---|---|---|---|---|
| Larva | T183 | U2 | 239.4 | 207.5 | 31.9 | 13.8 | 14.6 | <0.0001* |
| Larva | BL019A | U10 | 233.3 | 212.1 | 21.2 | 5.6 | 6.6 | 0.0002* |
| Larva | SATT302 | U10 | 234.4 | 211.9 | 22.5 | 6.2 | 6.8 | 0.0002* |
| Pupa | A089 | U10 | 249.6 | 237.0 | 12.6 | 8.4 | 9.3 | <0.0001* |
| Pupa | SATT302 | U10 | 249.3 | 239.0 | 10.3 | 5.4 | 5.9 | 0.0005* |

% Survival to

| | | | | | | | | |
|---|---|---|---|---|---|---|---|---|
| Pupal stage | R013 | U8 | 68.4 | 79.5 | −11.1 | 12.1 | 8.7 | <0.0001* |
| Pupal stage | BL019A | U10 | 68.0 | 60.5 | 7.5 | 5.2 | 6.1 | 0.0004 |

Larval developmental rate to⁵

| | | | | | | | | |
|---|---|---|---|---|---|---|---|---|
| Pupa | T183 | U2 | 5.394 | 5.211 | 0.183 | 8.7 | 10.1 | <0.0001* |
| Prepupa | BL019A | U10 | 6.092 | 5.881 | 0.221 | 7.4 | 8.7 | <0.0001* |

Nutritional efficiency index⁶

| | | | | | | | | |
|---|---|---|---|---|---|---|---|---|
| ECI, ECD | T183 | U2 | 15.7 | 13.6 | 2.2 | 6.8 | 7.8 | <0.0001* |
| ECI, ECD | S11 | U6 | 9.8 | 8.3 | 1.5 | 11.4 | 7.2 | <0.0001* |

¹Each QTL is indicated by the molecular marker to which it is linked
²Linkage group of marker (Utah linkage map of RI of the Noir 1 × Minsoy cross)
³LLR, Log likelihood ratio test, and P of LLR, probablity value based on 1 million runs, Monte Carlo (Chase et al. 1996);
*also significant by the sequential Bonferroni test, Dunn-Sidak method (Sokal and Rholf, 1995)
⁴Weight groups, Larva, 12 day larval weight; Pupa, pupal weight at 24-48 hrs after pupation
⁵Larval developmental rate, (1/number of days to reach indicated stage) × 100
⁶Nutritional efficiency measured during days 8-12 of larval development; ECI, ECD, % efficiency of conversion of ingested (ECI) or digested (ECD) food; CI, consumption index, see Table 1 and text for definitions

### EXAMPLE 2

RI soybean populations. The RI soybean populations and the genetic marker data have been described previously (Mansur and Orf, 1995, supra; Mansur et al., 1996, supra; Orf et al., 1999, supra). In brief, 240 RI lines were derived using a single seed descent from F2 segregants produced on reciprocal crosses of ‘Noir 1’ (PI 290136) and ‘Minsoy’ (PI 27890) (MN population). F13 or F14 generation seed stock was used from the MN population for the bioassay experiments. A new RI population was derived from reciprocal crosses of the parents, Archer (an elite cultivar) and Minsoy (MA RI population) using similar methods (Orf et al., 1999). The MA population has been advanced to the F10 generation and has 233 RI segregants. F10 generation seed stock was used from the MA population for the bioassay experiments.

Genetic markers for mapping within the RI population consisted of 150 restriction fragment length polymorphisms (RFLPs), 238 simple sequence repeat markers (SSRs) and two of the soybean classical morphological markers. Procedures for developing these markers have been detailed previously (Apuya et al., 1988, supra; Keim and Shoemaker, 1988, supra; Akkaya et al., 1992, supra; Lark et al., 1993, supra; Cregan et al., 1994a, b, supra; Akkaya et al., 1995, supra; Mansur et al., 1996, supra). Minsoy and Noir 1 were screened for RFLP polymorphisms. Polymorphic probes were then hybridized against Southern blots of restriction fragments of DNA from RI lines. Primer sequences of polymorphic SSR loci have been presented elsewhere (Mansur et al., 1996, supra; Cregan et al., 1999, supra). From the genetic marker data set, a composite genetic map of the RI populations (the two RI described above as well as a population derived from a cross of Noir 1 X Archer) was prepared (Cregan et al., 1999, supra; Orf et al., 1999, supra). Linkage of loci and mapped distances were determined using two programs, Mapmaker 3.0 (Lander et al., 1987, supra; Lincoln and Lander, 1993, supra) and Join-Map (Stam, 1993, supra) as reported by Cregan et al. (1999), supra. RFLP markers were developed during the F9 generation. Heterozygous lines were eliminated from the QTL analysis. No distorted segregation ratios were observed for alleles of any RFLP or SSR marker in either RI population.

Herbivore bioassays were carried out as described in Example 1.

All 240 of the MN RI and 228 of the 233 MA RI were tested. Resistant and susceptible RI controls from the MN population were included in each experiment. Across all tests and both RI populations, the larval weight of the resistant control was highly correlated with values of the average for RIs of each test (r=0.82 to 0.86, P<0.0001) and with the susceptible standard (r=0.74 to 0.79, P<0.001), and correlations between the susceptible control and the average larval weight values of RI in each test were high (r=0.78 to 0.83), suggesting relative homogeneity of the results among tests. In addition, 104 RI lines of the MN population were also tested on another H. zea culture from the North Carolina State University Rearing Facility. Similar resistance rankings were observed among the repeated lines, especially of the standards used (Terry, et al. (1999) Entomol. Exp. Appl. 191:465-476) which indicates consistency in the estimation of resistance.

Determination of linkage of traits to genetic markers and statistical analyses. Normalized trait values were used to identify plant QTLs. Although plant stage and bioassay conditions were similar across all experiments, average larval weights at 12 days after hatch and developmental rates varied from test to test, possibly due to variations in plant growth and greenhouse conditions across seasons. Therefore, trait values within each test were standardized by normalizing the data within each test, e.g., normalized larval weight=(larval weight—average larval weight for test)/each test's weight standard deviation. These adjusted trait values are correlated with their respective trait's unadjusted data (r=0.57 to 0.66, P<0.0001). This normalization procedure minimizes effects of environmental variation.

Transgressive segregation for each trait within each RI population was determined by calculating whether the number of RI outside the combined parental 95% confidence limits for each trait exceeded the number predicted by chance (P<0.05, binomial distribution).

A simple interval mapping feature of the computer package PLABQTL (Utz, H. F. and Melchinger, A. E., (1996) J. Quant. Loc. was used for detecting QTL. This program uses a multiple regression approach to interval mapping with marker order and distances determined by Mapmaker. We established empirical LOD thresholds for QTL detection using permutation tests (Churchill, G. A. and Doerge, R. W. (1994) Genetics 138:963-971). A LOD of ≧3.8 has an experiment-wise significance of P≦0.05. The PLABQTL program was used to perform a simultaneous fit of all QTL detected above a threshold of 2.5. All QTL detected above a threshold LOD of 2.5. However, where LOD score peaks are broad across linked markers, it is difficult to exactly locate QTLs in these intervals (see discussion by Liu, B. H., Statistical Genomics: Linkage, Mapping, and QTL analysis, (1998) CRC Press LLC, Boca Raton, 611 pp.), and more fine scale mapping is needed to determine if there is one or more QTL and their locations. The total amount of variation explained by the simultaneous fit of all significant QTLs and that due to each parent was calculated from the partial sums of squares using the PLABQTL program. In these data, significant loci on the same linkage group were considered as separate QTL if they were >50 cM apart, if markers between had significantly lower LOD scores and probability value (Mansur et al., 1996, supra), if they affect different traits, or if the parental origin of the resistant allele reversed from one marker to the other.

Direct effects of each QTL identified by interval mapping were determined using EPISTAT (Chase et al., 1997), a program that uses Maximum Likelihood (ML) models to determine the effect associated with a marker (QTL) as opposed to its occurrence by random chance (null model). The individual effects of each QTL are reported as r² values, and the parental allele associated with resistance is indicated. Monte Carlo simulations (1 million runs each) were used to calculate the probability of the direct effect of each marker.

Lines within a set of tests were selected randomly. A close to 1:1 segregation of alleles was found in almost all sets of tests in each population for the significant QTL, thereby minimizing the potential for false positive identification of QTL due to segregation ratio distortion.

Analyses of variance (GLM models, SAS Institute Inc., 1988) were used to obtain genetic and error variance components to calculate the broad sense heritability estimates (Burton, G. W. and DeVane, E. H. (1953) Agron. J. 45:478-481). The genetic and error variance components for each population were obtained from the pooled sums of squares for genotype and for error across all sets of RI lines divided by their respective pooled degrees of freedom.

TABLE 6
Means, standard errors (SE) and ranges of the corn earworm larval developmental
traits measured when reared on the two RI soybean populations, Minsoy × Noir 1 or Minsoy ×
Archer.

| | Minsoy × Archer | | | Minsoy × Noir 1 | | | Archer | Minsoy | Noir 1 |
|---|---|---|---|---|---|---|---|---|---|
| Trait | Mean | SE | Range | Mean | SE | Range | Mean | Mean | Mean |
| Larval Weight (mg) | 234.7 | 5.5 | 63-433 | 228.7 | 4.4 | 59-385 | 270.0 | 191.0 | 255.1 |
| Pupal Weight (mg) | 270.0 | 1.4 | 183-346 | 245.9 | 1.4 | 199-308 | 297.9 | 249.7 | 278.0 |
| Development Rate^{†} | 5.4 | 0.04 | 4-6.6 | 5.3 | 0.03 | 2.7-6.5 | 5.6 | 5.0 | 5.5 |
| % Survival to pupa | 85.5 | 1.1 | 25-100 | 74.0 | 1.0 | 26-100 | 95.0 | 80.0 | 85.0 |

^{†}Development rate, (Number of days to reach the pupal stage⁻¹) × 100

TABLE 7
Listing of all QTLs associated with corn earworm larval development
detected with a LOD > 2.4. The QTLs are grouped by RI population,
either Minsoy × Noir 1 or Minsoy × Archer. Within each population, each
QTL is characterized by the most significant linked marker; the linkage
group on which it is located; the P-value associated with the linkage; the
amount of variation explained by the QTL (R²); and which allele is
associated with resistance to larval development.

| | | | | | |
|---|---|---|---|---|---|
| | | Linkage | | | Resistant |
| Trait | Marker | group | P-value ^{†} | R² (%)^{‡} | allele ^{§} |

Minsoy × Noir 1 RI

| | | | | | |
|---|---|---|---|---|---|
| Larval weight | Sat_112 | U2 | <0.00001 | 17.0 | M |
| Larval weight | Satt353 | U10 | 0.00052 | 5.3 | M |
| Larval weight | Satt192 | U10 | 0.00048 | 5.5 | M |
| Larval weight | Satt302 | U10 | 0.00004 | 7.6 | M |
| Larval weight | L204_2 | U12 | 0.00168 | 5.6 | N |
| Pupal weight | Sat_112 | U2 | 0.00002 | 9.3 | M |
| Pupal weight | Satt192 | U10 | 0.00061 | 5.5 | M |
| Development rate | A060_1 | U1 | 0.00087 | 4.8 | N |
| Development rate | Sat_112 | U2 | <0.00001 | 12.0 | M |
| Development rate | Satt192 | U10 | 0.00012 | 6.8 | M |
| Development rate | Satt302 | U10 | <0.00001 | 9.4 | M |
| Development rate | L204_2 | U12 | 0.00289 | 5.0 | N |
| Survival | R013_2 | U8 | 0.00002 | 8.0 | N |
| Survival | Satt507 | U8 | 0.00015 | 7.0 | N |

Minsoy × Archer RI

| | | | | | |
|---|---|---|---|---|---|
| Larval weight | Satt575 | U2 | <0.00001 | 29.0 | M |
| Pupal weight | Satt575 | U2 | 0.0002 | 8.0 | M |
| Pupal weight | Satt567 | U11 | 0.00035 | 7.0 | M |
| Development rate | Satt575 | U2 | <0.00001 | 28.0 | M |
| Development rate | Satt365 | U9 | 0.00022 | 7.0 | M |
| Survival | Satt575 | U2 | 0.00228 | 5.0 | M |

^{†} P value estimated from Monte Carlo simulations, 1 million runs for each QTL
^{‡} R² values determined from one-way ANOVA for each QTL
^{§} N = Noir1 allele; M = Minsoy allele

TABLE 8
Summary statistics for the analysis of corn earworm larval
development traits in the RI populations. For each trait
within each RI population, the heritability (H²);
the percent of variation (VQTL) explained by all the QTL in
total, and that due to either Minsoy, Archer or Noir (R²);
the number of QTL detected with LOD > 4.0; and the number
of QTL explaining >10% of the variation (QTL with R² > 10%) are listed.
For specific distances and ordering of markers see FIG. 2.

| | | VOTL ^{†} | | | # QTL | # QTL |
|---|---|---|---|---|---|---|
| Trait | H² | Total | M ^{‡} | A or N ^{‡} | LOD > 4.0 | R² > 10% |

Minsoy × Archer RI

| | | | | | | |
|---|---|---|---|---|---|---|
| Larval weight | 54 | 27 | 27 | 0 | 1 | 1 |
| Pupal weight | 65 | 13 | 13 | 0 | 0 | 0 |
| Development | 46 | 36 | 36 | 0 | 1 | 1 |
| rate | | | | | | |
| Survival | 58 | 0 | — | — | 0 | 0 |

Minsoy X Noir 1 RI

| | | | | | | |
|---|---|---|---|---|---|---|
| Larval weight | 55 | 33 | 27 | 6 | 2 | 1 |
| Pupal weight | 48 | 14 | 14 | 0 | 1 | 0 |
| Development | 42 | 33 | 27 | 6 | 3 | 1 |
| rate | | | | | | |
| Survival | 54 | 14 | 0 | 14 | 1 | 0 |

^{†} VQTL R² values based upon partial sums of squares, PLABQTL multiple regression program
^{‡} VQTL due to M, Minsoy allele; or due to either A, Archer, or N, Noir 1 allele, depending upon the RI population

What is claimed is:

1. A method of breeding a plant to affect an interaction between a plant and an interacting organism comprising the steps of:
(a) crossing a first parental plant line and a second parental plant line to give a plurality F1 progeny;
(b) selfing individuals of said plurality of F1 progeny to give a population of recombinant inbred plant lines;
(c) assaying the affect of said first parental plant line, said second parental plant line, and individuals of said population of recombinant inbred plant lines on an interacting organism;
wherein an interaction between a plant and an interacting organism is affected when individuals of said recombinant inbred plant lines assay differently than said first parental plant varietal and said second plant parental varietal.

2. The method of claim 1 wherein the first parental plant line and the second parental plant line are substantially genetically different.

3. The method of claim 1 wherein the population of recombinant inbred plant lines and the first parental plant line and the second parental plant lines are mapped with molecular markers.

4. The method of claim 3 wherein the molecular markers are SSR markers or predominantly SSR markers.

5. The method of claim 1 wherein the plant is selected from the group consisting of sugar cane, wheat, rice, soybean, maize, potato, sugar beet, cassava, barley, soybean, sweet potato, oil palm fruit, tomato, sorghum, orange, grape, banana, apple, cabbage, watermelon, coconut, onion, cottonseed, rapeseed, and yam.

6. The method of claim 5 wherein the plant is soybean.

7. The method of claim 6 wherein the first parental plant line and the second parental plant line are selected from the group consisting of Noir 1, Minsoy, and Archer.

8. The method of claim 3 wherein said interaction between a plant and an interacting organism is affected by a quantitative trait loci linked to a molecular marker.

9. The quantitative trait loci identified by the method of claim 9.

10. The quantitative trait loci of claim 9.

11. A quantitative trait loci which affects an interaction between a plant and an interacting organism, and is linked to the an SSR selected from the group consisting of Satt192, Satt285, Satt301, Satt302, Satt353, Satt507, Satt531, Satt575, Sct_{—}046, and Sat_{—}112.

12. A SSR linked to a quantitative trait loci, wherein said SSR has the following pairs of primers
SEQ ID NO:1 and SEQ ID NO:2;
SEQ ID NO:3 and SEQ ID NO:4;
SEQ ID NO:5 and SEQ ID NO:6;
SEQ ID NO:7 and SEQ ID NO:8;
SEQ ID NO:9 and SEQ ID NO:10;
SEQ ID NO:11 and SEQ ID NO:12;
SEQ ID NO:13 and SEQ ID NO:14;
SEQ ID NO:15 and SEQ ID NO:16;
SEQ ID NO:17 and SEQ ID NO:18; and
SEQ ID NO:19 and SEQ ID NO:20.

13. A method of breeding a plant with an improved quantitative trait of agronomic value comprising the steps of:
(a) selecting a first parental plant line and a second parental plant line;
(b) crossing said first parental plant line and said second parental plant line to give a plurality of F1 plants;
(c) preparing a plurality of recombinant inbred plant lines from individuals of said plurality of F1 plants obtained from step (b);
(d) assaying said first parental plant line, said second parental plant line, and said plurality of recombinant inbred plant lines for a quantitative trait of agronomic value;
wherein a plant with an improved quantitative trait is identified when an said individual of said plurality of progeny plant lines performs better in said assay for said quantitative trait of agronomic value than either said first parental plant line or said second parental plant line.

14. The method of claim 13 wherein the first parental plant line and the second parental plant line are substantially genetically different.

15. The method of claim 13 wherein the population of recombinant inbred plant lines and the first parental plant line and the second parental plant line are mapped with molecular markers.

16. The method of claim 15 wherein the molecular markers are SSR markers or predominantly SSR markers.

17. The method of claim 16 wherein the plant is selected from the group consisting of sugar cane, wheat, rice, soybean, maize, potato, sugar beet, cassava, barley, soybean, sweet potato, oil palm fruit, tomato, sorghum, orange, grape, banana, apple, cabbage, watermelon, coconut, onion, cottonseed, rapeseed, and yam.

18. The method of claim 17 wherein the plant is soybean.

19. The method of claim 18 wherein the first parental plant line and the second parental plant line are selected from the group consisting of Noir 1, Minsoy, and Archer.

20. The method of claim 19 wherein said interaction between a plant and an interacting organism is affected by a quantitative trait of agronomic value linked to a molecular marker.

21. A quantitative trait of agronomic value identified by the method of claim 20.

22. A method of breeding a plant quantitative trait loci into a different plant line comprising the steps of:
(a) identifying a quantitative loci according to the method of claim 1; and
(b) introgressing said quantitative trait loci into said different plant.

23. The method of claim 22 wherein the quantitative trait loci affects an interaction between a plant and an interacting organism.

24. The method of claim 23 wherein the plant is soybean and the interacting organism is a soybean pest.

25. The method of claim 24 wherein the soybean pest is selected from the group consisting of beetle, bean leaf beetle, blister beetle, spotted cucumber beetle, grape colaspis beetle, Japanese beetle, Mexican bean beetle; caterpillars, armyworms (beet and yellow striped), corn earworms, green cloverworm, soybean looper, velvetbean, Mexican bean beetle larva; stink bugs, brown and green/Southern green stink bugs, orange colaspis larvae; soybean stem borer, three-cornered alfalfa hopper, viruses, bacteria, fungi, microorganisms.

26. The method of claim 23 wherein said quantitative trait loci is linked to a molecular marker.

27. The method of claim 26 wherein said molecular marker is an SSR or RFLP.

28. The method of claim 27 wherein the SSR is selected from the group consisting of Satt192, Satt285, Satt301, Satt302, Satt353, Satt507, Satt531, Satt575, Sct_{—}046, and Sat_{—}112.
